(19) United States
(12) Patent Application Publication
(10) Pub. No.: US 2023/0355656 A1
(43) Pub. Date: Nov. 9, 2023

(54) COMPOSITIONS AND METHODS FOR TREATMENT OF SKIN CANCERS

(71) Applicant: Sirnaomics, Inc., Germantown, MD (US)

(72) Inventors: Michael MOLYNEAUX, Germantown, MD (US); David M. EVANS, Germantown, MD (US); Patrick Y. LU, Germantown, MD (US)

(21) Appl. No.: 18/355,637

(22) Filed: Jul. 20, 2023

Related U.S. Application Data
(63) Continuation of application No. PCT/US2022/013426, filed on Jan. 21, 2022.
(60) Provisional application No. 63/140,018, filed on Jan. 21, 2021.

(51) Int. Cl. A61K 31/713; A61P 35/00; A61K 9/51; C07K 16/28

(52) U.S. Cl. CPC A61K 31/713; A61P 35/00; A61K 9/5146; C07K 16/2818; C07K 16/2827; C07K 16/2803; A61K 2039/505

(57) ABSTRACT

siRNA sequences for inhibiting TGFβ and Cox-2 gene expression are provided. Methods for treatment of skin cancers, in which pharmaceutical compositions or containing these siRNA agents and complexes, are further provided, in particular, for treating squamous cell carcinoma (isSCC) and/or basal cell carcinoma (BCC). TGFβ and Cox-2 have each been implicated in driving cancer progression. TGFβ is upregulated in a number of tumor types and plays a role in stimulating cancer-associated fibroblast development. Cox-2 upregulation plays a negative role in inducing inflammation and converting active T-cells to inactive T-reg cells. Co-delivery of the two siRNAs into the same cell at the same time silences of both targets at the same time results in antitumoral activity.

### RELATED APPLICATIONS

This application is a continuation of International Application No. PCT/US22/13426, filed Jan. 21, 2022, which claims the benefit of and priority to U.S. Provisional Application No. 63/140,018, filed Jan. 21, 2021, the contents of each of which are incorporated herein by reference in their entireties.

### SEQUENCE LISTING

The instant application contains a Sequence Listing submitted electronically in ST.26 (XML) format and hereby incorporated by reference in its entirety. The XML file, created on Jul. 20, 2023, is named 4690.0035C ST.26 Sequence Listing and is 135,218 bytes in size.

### FIELD

RNAi agents for inhibiting TGFβ and Cox-2 gene expression are provided. Methods for treatment of skin cancers, in which pharmaceutical compositions or containing these RNAi agents and complexes, are further provided, in particular, for treating squamous cell carcinoma (isSCC) and/or basal cell carcinoma (BCC).

### BACKGROUND

TGFβ and Cox-2 have each been implicated in driving cancer progression. TGFβ is upregulated in a number of tumor types and plays a role in stimulating cancer-associated fibroblast development. Cox-2 upregulation plays a negative role in inducing inflammation and converting active T-cells to inactive T-reg cells. Previously it was shown that administration of two siRNAs targeting TGFβ1 or Cox-2 in a single nanoparticle formulation allows co-delivery of the two siRNAs into the same cell at the same time, and that silencing of both targets at the same time results in antitumoral activity.

### SUMMARY

Methods of using pharmaceutical compositions comprising siRNA targeting TGFβ1 and COX-2 to treat skin cancers are provided. In some embodiments methods of treatment are provided for treating squamous cell carcinomas (isSCC) or basal cell carcinomas (BCC) by administering to a patient suffering from isSCC and/or BCC an effective amount of a nanoparticle formulation comprising at least one siRNA that inhibits the activity of TGFβ1 and at least one siRNA that inhibits Cox-2.

In other embodiments the nanoparticle formulation comprises HKP and/or HKP(+H). In still other embodiments the nanoparticle formulation is administered through intra-tumoral injection or IV (systemic) administration.

In other embodiments the formulation product is administered together with an immune checkpoint inhibitor. In certain embodiments the immune checkpoint inhibitor is an antibody or other agent that binds or inhibits a checkpoint protein selected from the group consisting of PD-1, PDL1, Lag3, Tim3, and CTLA-4/B7.

In other embodiments the immune checkpoint inhibitor is a PD-1 inhibitor. In certain of these embodiments the PD-1 inhibitor is selected from the group consisting of Pembrolizumab (Keytruda), Nivolumab (Opdivo), and Cemiplimab (Libtayo).

In other embodiments the immune checkpoint inhibitor is a PD-L1 inhibitor. In certain of these embodiments the PD-1 inhibitor is selected from the group consisting of Atezolizumab (Tecentriq), Avelumab (Bavencio), and Durvalumab (Imfinzi). In other embodiments the immune checkpoint inhibitor is a CTLA-4 inhibitor; in certain of these embodiments the CTLA-4 inhibitor is Ipilimumab (Yervoy).

In other embodiments the immune checkpoint inhibitor is a lymphocyte activation gene-3 (LAG-3) inhibitor. In some of these embodiments the LAG-3 inhibitor is BMS-986016.

In other embodiments the immune checkpoint inhibitor targets T cell immunoglobulin and mucin-domain containing-3 (TIM-3), T cell immunoglobulin and ITIM domain (TIGIT), V-domain Ig suppressor of T cell activation (VISTA), B7 homolog 3 protein (B7-H3) or B and T cell lymphocyte attenuator (BTLA)).

In other method embodiments, a pharmaceutical composition is used to treat basal cell carcinoma (BCC), comprising administering to a patient suffering from BCC an effective amount of a pharmaceutical composition comprising at least one siRNA that inhibits the activity of TGFβ1 and at least one siRNA that inhibits Cox-2, wherein the composition is administered in one embodiment to the patient in a dosage of between about 20 and 120 μg, at least once weekly for between about 1 and 12 weeks; under this treatment, tumor growth of the BCC in the patient is attenuated or inhibited.

In still other embodiments the pharmaceutical composition is administered to the patient in a dosage ranging between about 5 and about 170 μg, between about 10 and about 160 μg, between about 10 and about 130 μg, between about 10 and about 70 μg, between about 10 and about 40 μg, between about 20 and about 50 μg, between about 20 and about 30 μg, between about 30 and about 70 μg, between about 40 and about 80 μg, between about 60 and about 90 μg, between about 50 and about 100 μg, between about 70 and about 100 μg, and between about 80 and about 120 μg, at least once weekly for one to 12 weeks.

In other embodiments a local skin response in the patient is reduced following treatment. In still other embodiments, a histological clearance of BCC in the patient is dose-dependent.

### BRIEF DESCRIPTION OF THE FIGURES

FIG. 1 shows the highly significant reduction of target mRNA gene expression by STP705 (with siRNAs TGFβ1 and Cox-2) and downstream effects on select targets including αSMA, Col1A1 and Col3A1. The siRNAs are packaged in histidine-lysine polymer-(HKP-) containing nanoparticles.

FIG. 2 shows the significant reduction in TGFβ1 mean H-score (w SEM) when STP705 is administered to humans with isSCC.

FIG. 3 shows the reduction in COX-2 mean H-score (w SEM) when STP705 is administered to humans with isSCC.

FIG. 4 shows the pre- and post-treatment measurements of CD4+ and CD8+ cells both with residual tumor (a) and without it (b) in patients with isSCC. Although there was not a significant effect, there appears to be a tendency for an increase in T-cell penetration in subjects with residual tumor, but no effect in those without residual tumor.

FIG. 5 shows the significant reduction in Ki-67 cell proliferation protein expression (mean H-score±SEM) following administration (at 10-30 μg) of STP705 to humans with isSCC. Ki67 staining was performed to measure proliferating cells.

FIG. 6 shows the significantly reduced expression (p<0.031) of LC3B autophagy marker within the tumor site following STP705 administration (at 10-30 μg) in humans with siSCC.

FIG. 7 shows the significant effect (p=0.022) of STP705 administration (10-30 μg) on NFkB protein mean H-score (±SEM) in humans with siSCC levels.

FIG. 8 shows the significant effect on mean H-score (±SEM) Beta-Catenin levels within the tumor following STP705 administration (at 10-30 μg) in humans with isSCC.

FIG. 9 shows the significant dose-dependent response in Beta catenin level (as mean H-score (±SEM)) following STP705 administration (at 10, 20 and 30 μg) post-treatment.

FIG. 10 shows the significant attenuation of the increase in tumor size over time following administration of (i) high (40 μg) and (ii) low (20 μg) dose STP705 and (iii) DPP in phase II clinical trials in humans with isSCC.

FIG. 11 shows significantly reduced tumor weight at the end of the study in isSCC patients following each of (i) high (40 μg), (ii) low dose (20 μg) STP705 and (iii) DPP.

FIG. 12 shows maintenance of body weight in STP705-treated subjects in the two weeks post-treatment, and the loss of body weight in subjects administered DDP during the latter part of that period.

FIG. 13 shows a table with preliminary results of a clinical study of the effects of STP705 in patients to treat BCC in three cohorts (n=5), each receiving either 30, 60 or 90 μg doses.

### DETAILED DESCRIPTION

Compositions and methods for treating in situ Squamous cell Carcinoma (isSCC) and/or basal cell carcinoma (BCC) are provided. The compositions comprise at least one siRNA that inhibits the activity of TGFβ1 and at least one siRNA that inhibits Cox-2. Advantageously, the formulation is a nanoparticle formulation and may contain, for example, HKP and/or HKP(+H). The formulation may be administered through intra-tumoral injection or through intravenous (systemic) administration. The formulation may be administered together with an immune checkpoint inhibitor. Advantageously the immune checkpoint inhibitor is an antibody or other agent that binds or inhibits a checkpoint protein selected from the group consisting of PD-1, PDL1, Lag3, Tim3, and CTLA-4/B7. The checkpoint inhibitor may be, for example: a PD-1 inhibitor, such as Pembrolizumab (Keytruda), Nivolumab (Opdivo), or Cemiplimab (Libtayo); a PD-L1 inhibitor such as Atezolizumab (Tecentriq), Avelumab (Bavencio), and Durvalumab (Imfinzi); a CTLA-4 inhibitor such as Ipilimumab (Yervoy); a lymphocyte activation gene-3 (LAG-3) inhibitor such as BMS-986016; or may be an immune checkpoint inhibitor that targets T cell immunoglobulin and mucin-domain containing-3 (TIM-3), T cell immunoglobulin and ITIM domain (TIGIT), V-domain Ig suppressor of T cell activation (VISTA), B7 homolog 3 protein (B7-H3) or B and T cell lymphocyte attenuator (BTLA)).

We previously demonstrated that administration of TGFβ1 siRNA and Cox-2 siRNA in the peptide nanoparticle consisting of a branched histidine lysine copolymer that we could demonstrate efficacy of the combination in treating wounds as well as resolving hypertrophic scars. Zhou et al., Oncotarget 8: 80651-80665 (2017). We demonstrated that co-delivery of the 2 siRNAs simultaneously silencing TGFβ1 and Cox-2 resulted in human fibroblast apoptosis (Id.). Furthermore, HKP (histidine lysine branched polymer) formed nanoparticles with the siRNA and served to protect the siRNAs from degradation when administered in vivo and also allowed uptake of the two siRNAs into the same cells at the same time. I t was shown that HKP mediated siRNA delivery into human hypertrophic scars and the product resulted in a significant reduction in the size of the hypertrophic scars. This was further translated into a reduction of the size of human skin grafts administered to the mice. The mechanism was through an antifibrotic action on the skin samples.

In a clinical trial as described herein the nanoparticle formulation was administered to tumors in patients with in situ Squamous Cell Carcinoma (isSCC). The product was administered by direct intra-tumoral injection at doses of 10, 20, 30, 60 or 120 μg. Six (6) doses were administered per tumor on a weekly basis. Clinical results of this trial demonstrated a significant dose-dependent effect of the treatment in reducing the volume of tumor and, at doses of 30 μg, 60 μg, and 120 μg per dose, clinical clearance of the lesions in 13 of 15 (87%) of patients with the tumors was observed.

IHC staining of samples recovered from biopsies of the tumors post administration of the compound suggested that the rationale for the increase in efficacy through administration of both TGFβ1 and Cox-2 siRNAs was through an increase in recruitment of CD4+ and CD8+ T-cells into the solid tumor. This effect is augmented by reducing the TGFβ gradient that occurs surrounding tumors and that has been demonstrated to inhibit penetration of T-cells into the tumors (Daniele et al., Nature 554:538-546 (2018); Mariathasan et al., Nature 554:544-548 (2018)). Elevated Cox-2 also plays a role in inhibiting active T-cell recruitment to tumors (Gao et al., Digestion 79:169-76 (2009)). Inhibition of Cox-2 expression within the tumor microenvironment is expected to inhibit the conversion of active T-cells into Tregs (Id.)—augmenting the activity of the recruited T-cells. Therefore, the combination treatment has a surprising and dramatic effect in recruiting T-cells and maintaining their ability to fight against non-self cells (tumor cells).

The sequence of the sense strand of the TGFβ1 and Cox-2 siRNAs are shown below in Table 1 along with the sequences of the same genes in humans, mice, monkeys and pigs. The siRNA sequences have identity to the genes in humans, mice and monkeys. Cox-2 siRNA also has identity with the gene in pigs. TGFβ1 siRNA has identity with the sequence in pig barring a single nucleotide (C-U). Tables 2 and 3 provide additional siRNA sequences against TGFβ1 and Cox-2.

TABLE 1

| | SEQ | | | SEQ. |
|---|---|---|---|---|
| | ID No | TGFβ1 | Cox-2 | ID No. |
| siRNA | 80 | 5′-CCCAAGGGCUACCAUGCCAACUUCU-3′ | 5′-GGUCUGGUGCCUGGUCUGAUGAUGU-3′ | 1 |
| Human | 81 | 5′-CCCAAGGGCUACCAUGCCAACUUCU-3′ | 5′-GGUCUGGUGCCUGGUCUGAUGAUGU-3′ | 2 |
| Mouse | 82 | 5′-CCCAAGGGCUACCAUGCCAACUUCU-3′ | 5′-GGUCUGGUGCCUGGUCUGAUGAUGU-3′ | 3 |
| Monkey | 83 | 5′-CCCAAGGGCUACCAUGCCAACUUCU-3′ | 5′-GGUCUGGUGCCUGGUCUGAUGAUGU-3′ | 4 |
| Pig | 84 | 5′-CCCAAGGGCUACCAUGCCAAuUUCU-3′ | 5′-GGUCUGGUGCCUGGUCUGAUGAUGU-3′ | 5 |

TABLE 2

| siRNA Sequence (TGFB and Cox2) | SEQ. ID No: |
|---|---|
| cccaagggcu accaugccaa cuucu | 6 |
| agaaguuggc augguagccc uuggg | 7 |
| ggucuggugc cuggucugau gaugu | 8 |
| acaucaucag accaggcacc agacc | 9 |
| gguggcugga acagccagau guguu | 10 |
| aacacaucug gouguuccag ccacc | 11 |
| gaggagccuu caggauuaca agauu | 12 |
| aaucuuguaa uccugaaggc uccuc | 13 |
| gcugacccug aaguucaucu gcauu | 14 |
| aaugcagaug aacuucaggg ucagc | 15 |
| ggauccacga gcccaagggc uacca | 16 |
| ugguagcccu ugggcucgug gaucc | 17 |
| cccaagggcu accaugccaa cuucu | 18 |
| agaaguuggc augguagccc uuggg | 19 |
| gagcccaagg gcuaccaugc caacu | 20 |
| aguuggcaug guagcccuug ggcuc | 21 |
| gauccacgag cccaagggcu accau | 22 |
| augguagccc uugggcucgu ggauc | 23 |
| cacgagccca agggcuacca ugcca | 24 |
| uggcauggua gcccuugggc ucgug | 25 |
| gaggucaccc gcgugcuaau ggugg | 26 |
| ccaccauuag cacgcgggug accuc | 27 |
| guacaacagc acccgcgacc gggug | 28 |
| cacccggucg cgggugcugu uguac | 29 |
| guggauccac gagcccaagg gcuac | 30 |
| guagcccuug ggcucgugga uccac | 31 |
| ggucuggugc cuggucugau gaugu | 32 |
| acaucaucag accaggcacc agacc | 33 |
| gagcaccauu cuccuugaaa ggacu | 34 |
| aguccuuuca aggagaaugg ugcuc | 35 |
| ccucaauuca gucucucauc ugcaa | 36 |
| uugcagauga gagacugaau ugagg | 37 |
| gauguuugca uucuuugccc agcac | 38 |
| gugcugggca aagaaugcaa acauc | 39 |
| gucuuugguc uggugccugg ucuga | 40 |
| ucagaccagg caccagacca aagac | 41 |
| gugccugguc ugaugaugua ugcca | 42 |
| uggcauacau caucagacca ggcac | 43 |
| caccauucuc cuugaaagga cuuau | 44 |
| auaaguccuu ucaaggagaa uggug | 45 |
| caauucaguc ucucaucugc aauaa | 46 |
| uuauugcaga ugagagacug aauug | 47 |
| gguggcugga acagccagau guguu | 48 |
| aacacaucug gcuguuccag ccacc | 49 |
| gcuggaacag ccagaugugu ugcca | 50 |
| uggcaacaca ucuggcuguu ccagc | 51 |
| cgccagauua ccaucugguu ucaga | 52 |
| ucugaaacca gaugguaauc uggcg | 53 |
| ggagcccggc aauuaugcca ccuug | 54 |
| caagguggca uaauugccgg gcucc | 55 |
| caaggauauc gaaggcuugc uggga | 56 |
| ucccagcaag ccuucgauau ccuug | 57 |
| ggacaagagg cgcaagaucu cggca | 58 |
| ugccgagauc uugcgccucu ugucc | 59 |
| gcaagaucuc ggcagccacc agccu | 60 |
| aggcuggugg cugccgagau cuugc | 61 |
| ccaucugguu ucagaaccgc cgggu | 62 |
| acccggcggu ucugaaacca gaugg | 63 |

TABLE 3

| | SEQ | | | SEQ. |
|---|---|---|---|---|
| dsRNA | ID NO: | Sense sequence | Antisense sequence | ID No. |
| hmTF-25-1 | 85 | 5′-r(GGAUCCACGAGCCCAAGGGCUACCA)-3′ | 5′-r(UGGUAGCCCUUGGGCUCGUGGAUCC)-3′ | 64 |
| hmTF-25-2 | 86 | 5′-r(CCCAAGGGCUACCAUGCCAACUUCU)-3′ | 5′-r(AGAAGUUGGCAUGGUAGCCCUUGGG)-3′ | 65 |
| hmTF-25-3 | 87 | 5′-r(CCUCAAUUCAGUCUCUCAUCUGCAA)-3′ | 5′-r(UUGCAGAUGAGAGACUGAAUUGAGG)-3′ | 66 |
| hmTF-25-4 | 88 | 5′-r(GAUCCACGAGCCCAAGGGCUACCAU)-3′ | 5′-r(AUGGUAGCCCUUGGGCUCGUGGAUC)-3′ | 67 |
| hmTF-25-5 | 89 | 5′-r(CACGAGCCCAAGGGCUACCAUGCCA)-3′ | 5′-r(UGGCAUGGUAGCCCUUGGGCUCGUG)-3′ | 68 |
| hmTF-25-6 | 90 | 5′-r(GAGGUCACCCGCGUGCUAAUGGUGG)-3′ | 5′-r(AGAAGUUGGCAUGGUAGCCCUUGGG)-3′ | 69 |
| hmTF-25-7 | 91 | 5′-r(GUACAACAGCACCCGCGACCGGGUG)-3′ | 5′-r(CACCCGGUCGCGGGUGCUGUUGUAC)-3′ | 70 |
| hmTF-25-8 | 92 | 5′-r(GUGGAUCCACGAGCCCAAGGGCUAC)-3′ | 5′-r(GUAGCCCUUGGGCUCGUGGAUCCAC)-3′ | 71 |
| hmCX-25-1 | 93 | 5′-r(GGUCUGGUGCCUGGUCUGAUGAUGU)-3′ | 5′-r(ACAUCAUCAGACCAGGCACCAGACC)-3′ | 72 |
| hmCX-25-2 | 94 | 5′-r(GAGCACCAUUCUCCUUGAAAGGACU)-3′ | 5′-r(AGUCCUUUCAAGGAGAAUGGUGCUC)-3′ | 73 |
| hmCX-25-3 | 87 | 5′-r(CCUCAAUUCAGUCUCUCAUCUGCAA)-3′ | 5′-r(UUGCAGAUGAGAGACUGAAUUGAGG)-3′ | 74 |
| hmCX-25-4 | 95 | 5′-r(GAUGUUUGCAUUCUUUGCCCAGCAC)-3′ | 5′-r(GUGCUGGGCAAAGAAUGCAAACAUC)-3′ | 75 |
| hmCX-25-5 | 96 | 5′-r(GUCUUUGGUCUGGUGCCUGGUCUGA)-3′ | 5′-r(UCAGACCAGGCACCAGACCAAAGAC)-3′ | 76 |
| hmCX-25-6 | 97 | 5′-r(GUGCCUGGUCUGAUGAUGUAUGCCA)-3′ | 5′-r(UGGCAUACAUCAUCAGACCAGGCAC)-3 | 77 |
| hmCX-25-7 | 98 | 5′-r(CACCAUUCUCCUUGAAAGGACUUAU)-3′ | 5′-r(AUAAGUCCUUUCAAGGAGAAUGGUG)-3′ | 78 |
| hmCX-25-8 | 99 | 5′-r(CAAUUCAGUCUCUCAUCUGCAAUAA)-3′ | 5′-r(UUAUUGCAGAUGAGAGACUGAAUUG)-3′ | 79 |

The siRNA molecules can produce additive or synergistic effects in the cells, depending on the compositions and structures of particular molecules. In a preferred embodiment, they produce a synergistic effect. Double-stranded RNA has been shown to silence gene expression via RNA interference (RNAi). Short-interfering RNA (siRNA)-induced RNAi regulation shows great potential to treat a wide variety of human diseases including from cancer.

RNAi is a sequence-specific RNA degradation process that provides a relatively easy and direct way to knockdown, or silence, theoretically any gene. In naturally occurring RNA interference, a double stranded (ds) RNA is cleaved by an endonuclease into siRNA molecules, overhangs at the 3′ ends. These siRNAs are incorporated into a multicomponent-ribonuclease called RNA-induced-silencing-complex (RISC). One strand of siRNA remains associated with RISC and guides the complex towards a cognate RNA that has sequence complementary to the guider single stranded siRNA (ss-siRNA) in RISC. This siRNA-directed endonuclease digests the RNA, thereby inactivating it. Studies have revealed that the use of chemically synthesized 21-25-nt siRNAs exhibit RNAi effects in mammalian cells, and the thermodynamic stability of siRNA hybridization (at terminals or in the middle) plays a central role in determining the molecule's function.

It is presently not possible to predict with a high degree of confidence which of many possible candidate siRNA sequences potentially targeting a mRNA sequence of a disease gene in fact exhibit effective RNAi activity. Instead, individually specific candidate siRNA polynucleotide or oligonucleotide sequences must be generated and tested in the mammalian cell culture to determine whether the intended interference with expression of a targeted gene has occurred. The unique advantage of siRNA makes it possible to be combined with multiple siRNA duplexes to target multiple disease-causing genes in the same treatment, since all siRNA duplexes are chemically homogenous with same source of origin and same manufacturing process.

As used herein, an “siRNA molecule” is a duplex oligonucleotide, that is a short, double-stranded polynucleotide, that interferes with the expression of a gene in a cell that produces RNA, after the molecule is introduced into the cell. For example, it targets and binds to a complementary nucleotide sequence in a single stranded (ss) target RNA molecule, such as an mRNA or a micro RNA (miRNA). The target RNA is then degraded by the cell. Such molecules are constructed by techniques known to those skilled in the art. Such techniques are described in U.S. Pat. Nos. 5, 898,031, 6,107,094, 6,506,559, 7,056,704 and in European Pat. Nos. 1214945 and 1230375. By convention in the field, when an siRNA molecule is identified by a particular nucleotide sequence, the sequence refers to the sense strand of the duplex molecule.

The siRNA molecule can be made of naturally occurring ribonucleotides, i.e., those found in living cells, or one or more of its nucleotides can be chemically modified by techniques known in the art. In addition to being modified at the level of one or more of its individual nucleotides, the backbone of the oligonucleotide can be modified. Additional modifications include the use of small molecules (e.g. sugar molecules), amino acid molecules, peptides, cholesterol, and other large molecules for conjugation onto the siRNA molecule.

In one embodiment, the molecule is an oligonucleotide with a length of about 19 to about 35 base pairs. In one aspect of this embodiment, the molecule is an oligonucleotide with a length of about 19 to about 27 base pairs. In another aspect, the molecule is an oligonucleotide with a length of about 21 to about 25 base pairs. In all these aspects, the molecule may have blunt ends at both ends, or sticky ends at both ends, or a blunt end at one end and a sticky end at the other.

In the composition of the disclosed embodiments, the relative amounts of the two different molecules and the copolymer can vary. In one embodiment, the ratio of the two different siRNA molecules is about 1:1 by mass. In another embodiment, the ratio of these molecules to the copolymer is about 1:4, 1:4.5, or 1:5 by mass. Preferably, the ratio of the two different siRNA molecules is about 1:1 by mass and the ratio of these molecules to the copolymer is about 1:4, 1:4.5, or 1:5 by mass. With these ratios, the composition forms nanoparticles with an average size of about 150 nm in diameter.

In one embodiment, the siRNA molecules are selected from those identified in any of Tables 1-3. An example is the pair designated hmTF-25-2 and hmCX-25-1 in Table 1, with the following sequences:

hmTF-25-2:
sense,
(SEQ ID NO: 86)
5′-r(CCCAAGGGCUACCAUGCCAACUUCU)-3′
antisense,
(SEQ ID NO: 65)
5′-r(AGAAGUUGGCAUGGUAGCCCUUGGG)-3′,
and
hmCX-25-1:
sense,
(SEQ ID NO: 93)
5′-r(GGUCUGGUGCCUGGUCUGAUGAUGU)-3′
antisense,
(SEQ ID NO: 72)
5′-r(ACAUCAUCAGACCAGGCACCAGACC)-3′.

The disclosed embodiments include a method for identifying the desired siRNA molecules comprising the steps of: (a) creating a collection of siRNA molecules designed to target a complementary nucleotide sequence in the target mRNA molecules, wherein the targeting strands of the siRNA molecules comprise various sequences of nucleotides; (b) selecting the siRNA molecules that show the highest desired effect against the target mRNA molecules in vitro; (c) evaluating the selected siRNA molecules in an animal wound model; and (d) selecting the siRNA molecules that show the greatest efficacy in the model for their silencing activity and therapeutic effect.

Importantly, it is presently not possible to predict with high degree of confidence which of many possible candidate siRNA sequences potentially targeting an mRNA sequence of a disease gene will, in fact, exhibit effective RNAi activity. Instead, individually specific candidate siRNA polynucleotide or oligonucleotide sequences must be generated and tested in mammalian cell culture, such as an in vitro organ culture assay, to determine whether the intended interference with expression of a targeted gene has occurred. The unique advantage of siRNA makes it possible to be combined with multiple siRNA duplexes to target multiple disease causing genes in the same treatment, since all siRNA duplexes are chemically homogenous with same source of origin and same manufacturing process.

Preferably, the siRNA molecules are evaluated in at least two of the animal models. In one embodiment, the method further includes the steps of adding a pharmaceutically acceptable carrier to each of the siRNA molecules to form pharmaceutical compositions and evaluating each of the pharmaceutical compositions in the animal wound model or models.

In one embodiment, the siRNA sequences are prepared in such way that each one can target and inhibit the same gene from, at least, both human and mouse, or human and non-human primate. In one aspect, the siRNA molecules bind to both a human mRNA molecule and a homologous mouse mRNA molecule. That is, the human and mouse mRNA molecules encode proteins that are substantially the same in structure or function. Therefore, the efficacy and toxicity reactions observed in the mouse disease models provide a good understanding about what is going to happen in humans. More importantly, the siRNA molecules tested in the mouse model are good candidates for human pharmaceutical agents. The human/mouse homology design of an siRNA drug agent can eliminate the toxicity and adverse effect of those species specificities observed in monoclonal antibody drugs.

In one embodiment, the disclosed embodiments provides a composition comprising two or more different siRNA molecules that bind to an mRNA that codes for TGFβ1 protein in a mammalian cell and two or more different siRNA molecules that bind to an mRNA that codes for COX-2 protein in a mammalian cell. The molecules may bind to different nucleotide sequences within the target mRNA. The siRNA molecules can produce additive or synergistic effects in the cells, depending on the compositions and structures of the particular molecules. In a preferred embodiment, they produce a synergistic effect. In certain applications of these embodiments, the siRNA molecules are selected from the ones identified in Tables 1-3.

The siRNA molecules are combined with a pharmaceutically acceptable carrier to provide pharmaceutical compositions for administering to a mammal. In the disclosed embodiments, the patient may be a mammal; the mammal may be a laboratory animal, such as a dog, cat, pig, non-human primate, or rodent, such as a mouse, rat, or guinea pig. Preferably, the mammal is a human.

Histidine-Lysine Co-Polymers. The carrier is a histidine-lysine copolymer that forms a nanoparticle containing an siRNA molecule, wherein the nanoparticle has a size of 100-400 nm in diameter. In one aspect of this embodiment, the carrier is selected from the group consisting of the HKP species, H3K4b and PT73, which have a Lysine backbone with four branches containing multiple repeats of Histidine, Lysine, or Asparagine. When an HKP aqueous was mixed with siRNA at a N/P ratio of 4:1 by mass, the nanoparticles (average size of 100-200 nm in diameter) were self-assembled. In another aspect of this embodiment, the HKP has the following formula:

(SEQ ID NO: 100)
(R)K(R)-K(R)-(R)K(X), where R = KHHHKHHHKHHHKHHHK,
or
(SEQ ID NO: 101)
R = KHHHKHHHNHHHNHHHN,
X = C(O)NH2, K = lysine, H = histidine, and
N = asparagine.

In still another aspect of this embodiment, the HKP has the following formula:

(SEQ ID NO: 100)
(R)K(R)-K(R)-(R)K(X), where R = KHHHKHHHKHHHKHHHK,
X = C(O)NH2, K = lysine, H = histidine.

In still another aspect of this embodiment, the HKP has the following formula: (HKP(+H)) where the third replicating HHHK motif (SEQ ID NO: 102) has an extra H (located 6 characters from the right end)

(SEQ ID NO: 103)
(R)K(R)-K(R)-(R)K(X), where R = KHHHKHHHKHHHHKHHHK,
X = C(O)NH2, K = lysine, H = histidine.

Pharmaceutical compositions of the disclosed embodiments are useful for down-regulating pro-fibrotic factors, such as a-smooth muscle actin (α-SMA), Hydroxyproline Acid, Smad 3, and Connective Tissue Growth Factor (CTGF), and fibrotic pathways, such as TGFβ1/Smad 3/α-SMA/Collagen I-III, in the cells of a tissue of a mammal. A therapeutically effective amount of the composition is administered to the tissue of the mammal. We hypothesized that using RNAi blocking the upstream factor of the pathway, such as TGFβ1, is a more potent inhibitor. Knowing the complicated network involved in this pathway, we hypothesized that inhibition of a related factor, such as Cox-2, in a different pathway may result in a synergistic effect for tighter control of the fibrosis pathway and its relevant network. In one embodiment, the tissue is skin scar, liver, lung, kidney, or heart tissue. In one aspect of this embodiment, the tissue is skin scar tissue. In another embodiment, the cells comprise fibroblasts and myofibroblasts. In one aspect of this embodiment, the fibroblasts and myofibroblasts are dermal fibroblasts and myofibroblasts.

Disclosed method embodiments comprising administering pharmaceutical compositions are also useful for activating fibroblast and myofibroblast apoptosis in the tissue of a mammal. This reduces tissue fibrosis caused by scarring after chronic inflammation of the tissue. Such apoptosis may be determined and measured by measuring the apoptotic cell population with FACS analysis, counting body numbers, and detecting expression levels of TGFβ1, Cox-2, α-SMA, Collagen I and Collagen III, Hydroxyproline acid, in vitro and in vivo.

One particular embodiment of the disclosed embodiments provides a method of activating fibroblast and myofibroblast apoptosis in a tissue of a human, comprising injecting into the tissue a therapeutically effective amount of a composition comprising the siRNA molecule hmTF-25-2: sense, 5′-r(CCCAAGGGCUACCAUGCCAACUUCU)-3′ (SEQ ID NO: 86), antisense, 5′-r(AGAAGUUGGCAUGGUAGCCCUUGGG)-3′ (SEQ ID NO: 65), the siRNA molecule hmCX-25-1: sense, 5′-r(GGUCUGGUGCCUGGUCUGAUGAUGU)-3′ (SEQ ID NO: 93), antisense, 5′-r(ACAUCAUCAGACCAGGCACCAGACC)-3′ (SEQ ID NO: 72), and a pharmaceutically acceptable carrier comprising a pharmaceutically acceptable histidine-lysine co-polymer. In one aspect of this embodiment, the histidine-lysine co-polymer comprises the histidine-lysine co-polymer species H3K4b or the histidine-lysine co-polymer species PT73. In another aspect of this embodiment, the histidine-lysine co-polymer has the formula:

(R)K(R)-K(R)-(R)K(X), where R=KHHHKHHHKHHHKHHHK (SEQ ID NO: 100), X═C(O)NH2, K=lysine, H=histidine, and N=asparagine. In still another aspect of this embodiment, the histidine-lysine co-polymer has the formula:

(SEQ ID NO: 100)
(R)K(R)-K(R)-(R)K(X), where R = KHHHKHHHKHHHKHHHK,
or
(SEQ ID NO: 103)
R=KHHHKHHHKHHHHKHHHK,
X = C(O)NH2, K = lysine, H = histidine.

Another disclosed embodiment provides a method for treatment of BCC or isSCC in a mammal, comprising injecting into the tissue a therapeutically effective amount of a composition comprising the siRNA molecule hmTF-25-2: sense, 5′-r(CCCAAGGGCUACCAUGCCAACUUCU)-3′ (SEQ ID NO: 86), antisense, 5′-5 r(AGAAGUUGGCAUGGUAGCCCUUGGG)-3′ (SEQ ID NO: 65), the siRNA molecule hmCX-25-1: sense, 5′-r(GGUCUGGUGCCUGGUCUGAUGAUGU)-3′ (SEQ ID NO: 93), antisense, 5′-r(ACAUCAUCAGACCAGGCACCAGACC)-3′ (SEQ ID NO: 72), and a pharmaceutically acceptable carrier comprising a pharmaceutically acceptable histidine-lysine co-polymer. In one aspect of this embodiment, the histidine-lysine co-polymer comprises the histidine-lysine co-polymer species H3K4b or the histidine-lysine co-polymer species PT73. In another aspect of this embodiment, the histidine-lysine co-polymer has the formula:

(SEQ ID NO: 100)
(R)K(R)-K(R)-(R)K(X), where R = KHHHKHHHKHHHKHHHK,
X = C(O)NH2, K = lysine, H = histidine, and
N = asparagine.

In still another aspect of this embodiment, the histidine-lysine co-polymer has the formula:

(R)K(R)-K(R)-(R)K(X), where R=KHHHKHHHKHHHKHHHK (SEQ ID NO: 100), or with an additional H located 6 characters to the right end of this sequence:

(SEQ ID NO: 103)
R=KHHHKHHHKHHHHKHHHK,
X = C(O)NH2, K = lysine, H = histidine.

Other HKP copolymers suitable for use in the disclosed embodiments are provided in, e.g., U.S. Pat. Nos. 7,070,807; 7,163,695; 7,465,708; and 7,772,201.

A therapeutically effective amount of the pharmaceutical composition is delivered to the tissue. Such tissue includes, but is not limited to, skin, liver, lung, kidney, and heart tissue. The composition may be delivered by injection into the tissue, subcutaneous injection into the mammal, or intravenous injection into the mammal. In other embodiments the composition is administered topically. In still other embodiments, the composition is administered parenterally or orally.

It was previously demonstrated that siRNAs that inhibit TGFβ1 and Cox-2 expression can induce T-cell penetration into regions where their genes will be silenced and the expression of collagen will be reduced. Zhou et al. Oncotarget 8(46):80651-80665 (2017). As Jones et al. (2017) demonstrated, adipocytes upregulated several ECM-associated genes in mice after 20 and 34 weeks on a high fat diet, including TGFβ 1, inhba, itga5 and ctgf, the collagens (collal and col6a3), elastin (eln), fibronectin (fn1), and other TGFβ family members (Jones et al., 2017). TGFβ1 regulates gene expression through signaling or transcription factor pathways, including SMADs, JNK, ERKs and MRTFA/SRF. MRTFA was implicated as having a role in diet-induced metabolic disruption of adipose tissue by favoring fibrogenesis over adipogenesis. Id.

The disclosed embodiments provide a double stranded or single stranded nucleic acid that acts to silence the expression of a gene of interest. In the embodiments disclosed herein the siRNA or other nucleic acid molecules target and bind to complementary sequences on two target genes, TGFβ 1 and Cox-2, to silence both to elicit the desired therapeutic effect. In some embodiments the siRNA or other nucleic acid molecules are formulated together in a nanoparticle formulation with a polypeptide polymer containing at least one histidine residue and at least one lysine residue. More preferably in some embodiments, the polypeptide polymer is HKP or HKP(+H).

### Preparation of Formulations—Pharmaceutical Compositions

In certain embodiments, the disclosed embodiments provide for a pharmaceutical composition comprising the dsRNA agent of the disclosed embodiments. The dsRNA agent sample can be suitably formulated and introduced into the environment of the cell by any means that allows for a sufficient portion of the sample to enter the cell to induce gene silencing, if it is to occur. Many formulations for dsRNA are known in the art and can be used so long as the dsRNA gains entry to the target cells so that it can act. See, e.g., U.S. published patent application Nos. 2004/0203145 A1 and 2005/0054598 A1. For example, the dsRNA agent of the disclosed embodiments can be formulated in buffer solutions such as phosphate buffered saline solutions, liposomes, micellar structures, and capsids. Formulations of dsRNA agent with cationic lipids can be used to facilitate transfection of the dsRNA agent into cells. For example, cationic lipids, such as lipofectin (U.S. Pat. No. 5,705,188), cationic glycerol derivatives, and polycationic molecules, such as polylysine (published PCT International Application WO 97/30731), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier, such as HKPs, discussed infra. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be appreciated that the method of introducing dsRNA agents into the environment of the cell will depend on the type of cell and the make-up of its environment. For example, when the cells are found within a liquid, one preferable formulation is with a lipid formulation such as in lipofectamine and the dsRNA agents can be added directly to the liquid environment of the cells. Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA agents in a buffer or saline solution and directly inject the formulated dsRNA agents into cells, as in studies with oocytes. The direct injection of dsRNA agent duplexes may also be done. For suitable methods of introducing dsRNA, see U.S. published patent application No. 2004/0203145 A1.

In one embodiment, the siRNA molecule or other nucleic acid has a length of 19 to 27 base pairs of nucleotides; in another embodiment, the siRNA molecule or other nucleic acid has a length of 20 to 30 base pairs; in still another embodiment the siRNA molecule or other nucleic acid has a length of 24 to 28 base pairs. The molecule can have blunt ends at both ends, or sticky ends at both ends, or one of each. The siRNA molecule may include a chemical modification at the individual nucleotide level or at the oligonucleotide backbone level, or it may have no modifications. In one preferred embodiment an anti-TGFβ1 siRNA or anti-Cox-2 siRNA possesses strand lengths of 25 nucleotides. In another, an anti-TGFβ1 siRNA or anti-Cox-2 siRNA possesses strand lengths of 19 to 25 nucleotides. In some embodiments, the siRNA molecules can be asymmetric where one strand is shorter than the other (typically by 2 bases e.g. a 21mer with a 23mer or a 19mer with a 21mer or a 23mer with a 25mer). The strands may be modified by inclusion of a dTdT overhang group on the 3′ end of selected strands. See, e.g., Table 2 above.

Advantageously, the pharmaceutical composition is STP705. STP705 contains two siRNA oligonucleotides: TGF-β1-siRNA (STP705-1) and COX-2-siRNA (STP705-2), targeting TGF-β1 and COX-2 mRNA respectively. Each siRNA is double-stranded, 25 nucleotides long, and is blunt ended. The siRNA molecules are formulated with a peptide and trehalose. TGF-β1-siRNA is a small interfering nucleic acid that targets transforming growth factor β1.

The sense and antisense strands of the duplex that target TGFβ1 are:

ST-705-1S (Sense strand):
(SEQ ID NO: 6)
5′ CCC AAG GGC UAC CAU GCC AAC UUC U 3′
ST-705-1A (Antisense strand)
(SEQ ID NO: 7)
5′ AGA AGU UGG CAU GGU AGC CCU UGG G 3

The sense and antisense strands of the duplex that targets COX-2 are:

ST-705-2S (Sense strand):
(SEQ ID NO: 1)
5′ GGU CUG GUG CCU GGU CUG AUG AUG U 3′
ST-705-2A (Antisense strand)
(SEQ ID NO: 9)
5′ ACA UCA UCA GAC CAG GCA CCA GAC C 3′

Additional siRNA sequences are provided in Tables 1-3.

H3K4b is a branched peptide, with a backbone of three L-lysine residues, where the N-terminus and the three lysine ε-amino groups are linked to a histidine-lysine peptide chain with the structure KH3KH3KH3KH3 (SEQ ID NO: 104). The C-terminus of the peptide is amidated. The histidine-lysine copolymer carriers are further described infra.

The molecules are mixed with a pharmaceutically acceptable carrier to provide compositions for administering to a subject. Preferably, the subject is a human. In one embodiment, the composition comprises a pharmaceutically acceptable carrier and at least three siRNA molecules, wherein each siRNA molecule binds an mRNA molecule that encodes a gene selected from the group consisting of pro-inflammatory pathway genes, pro-angiogenesis pathway genes, and pro-cell proliferation pathway genes. In still another embodiment, each siRNA contains at least three siRNA duplexes that target at least three different gene sequences. Preferably, each gene is selected from a different pathway. The disclosed embodiments comprise pharmaceutically effective carriers for enhancing the siRNA delivery into the disease tissues and cells.

In various embodiments of the composition, the carrier comprises one or more components selected from the group consisting of a saline solution, a sugar solution, a polymer, a lipid, a cream, a gel, and a micellar material. Further components or carriers include: a polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer, biodegradable polyesters, such as poly(lactic acid) (PLA), poly(glycolic acid) (PGA), and poly(lactic-co-glycolic acid) (PLGA), PEG-PEI (polyethylene glycol and polyethylene imine), Poly-Spermine (Spermidine), and polyamidoamine (PAMAM) dendrimers. In preferred embodiments, the carrier is a histidine-lysine copolymer that is believed to form a nanoparticle containing an siRNA molecule, wherein the nanoparticle has a size of about 100 to 400 nm in diameter, or preferably 80 to 200 nm in diameter; and more preferably 80 to 150 nm in diameter. In some embodiments the siRNA molecule may be formulated with methylcellulose gel for topical administration. In some preferred embodiments, the nanoparticle, of a size ranging from 80 to 150, or 80 to 200 nm and containing an siRNA molecule, may be formulated for injection or infusion without methylcellulose gel. Methods of formulating nanoparticles with a methylcellulose gel are known in the art.

The phrase “pharmaceutically acceptable carrier” or “carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. The pharmaceutically acceptable carrier of the disclosed dsRNA compositions may be micellar structures, such as a liposomes, capsids, capsoids, polymeric nanocapsules, or polymeric microcapsules.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Modifications and Linkages. A dsRNA agent of the disclosed embodiments can be conjugated (e.g., at its 5′ or 3′ terminus of its sense or antisense strand) or unconjugated to another moiety (e.g., a non-nucleic acid moiety such as a peptide), an organic compound (e.g., a dye, cholesterol, or the like). Modifying dsRNA agents in this way may improve cellular uptake or enhance cellular targeting activities of the resulting dsRNA agent derivative as compared to the corresponding unconjugated dsRNA agent, are useful for tracing the dsRNA agent derivative in the cell or improve the stability of the dsRNA agent derivative compared to the corresponding unconjugated dsRNA agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al. Nucleic Acids Symposium Series 52: 133-4), and derivatives thereof.

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other, see, e.g., Usman and McSwiggen, supra; Eckstein, et al., International PCT Publication No. WO 92/07065; Usman et al, International PCT Publication No. WO 93/15187; Uhlman & Peyman. There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxy, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂—O-2′-bridge, 2′-LNA or other bicyclic or “bridged” nucleoside analog, and 2′-O--(N-methylcarbamate) or those comprising base analogs.

In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878. “Modified nucleotides” of the disclosed embodiments can also include nucleotide analogs as described above.

In reference to the nucleic acid molecules of the present disclosure, modifications may exist upon these agents in patterns on one or both strands of the ds ribonucleic acid (dsRNA). As used herein, “alternating” positions refers to a pattern where every other nucleotide is a modified nucleotide or there is an unmodified nucleotide (e.g., an unmodified ribonucleotide) between every modified nucleotide over a defined length of a strand of the dsRNA (e.g., 5′- 3; -3′-MNMNMN-5′; where M is a modified nucleotide and N is an unmodified nucleotide). The modification pattern starts from the first nucleotide position at either the 5′ or 3′ terminus according to a position numbering convention. The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. “Alternating pairs of positions” refers to a pattern where two consecutive modified nucleotides are separated by two consecutive unmodified nucleotides over a defined length of a strand of the dsRNA (e.g., 5′- MNN-3′; 3′-M MNNMMNNMMNN-5′; where M is a modified nucleotide and N is an unmodified nucleotide). The modification pattern starts from the first nucleotide position at either the 5′ or 3′ terminus according to a position numbering convention such as those described herein. The pattern of modified nucleotides at alternating positions may run the full length of the strand, but preferably includes at least 8, 12, 16, 20, 24, 28 nucleotides containing at least 4, 6, 8, 10, 12 or 14 modified nucleotides, respectively. It is emphasized that the above modification patterns are exemplary and are not intended as limitations on the scope of the disclosed embodiments.

As used herein, “loop” refers to a structure formed by a single strand of a nucleic acid, in which complementary regions that flank a particular ss nucleotide region hybridize in a way that the ss nucleotide region between the complementary regions is excluded from duplex formation or Watson-Crick base pairing. A loop is a ss nucleotide region of any length. Examples of loops include the unpaired nucleotides present in such structures as hairpins, stem loops, or extended loops.

An anti-TGFβ1 siRNA or anti-Cox-2 siRNA advantageously possesses strand lengths of 25 nucleotides.

In certain embodiments, the first and second oligonucleotide sequences of the siRNA or other nucleic acid exist on separate oligonucleotide strands that can be and typically are chemically synthesized. In some embodiments, both strands are 25 nucleotides in length, are completely complementary and have blunt ends. In certain embodiments of the disclosed embodiments, the anti-TGFβ1 siRNA or anti-Cox-2 siRNA exist on separate RNA oligonucleotides (strands). In certain embodiments TGFβ1 siRNA or anti-Cox-2 siRNA agent is comprised of two oligonucleotide strands of differing lengths, with one possessing a blunt end at the 3′ terminus of a first strand (sense strand) and a 3′ overhang at the 3′ terminus of a second strand (antisense strand). The siRNA can also contain one or more deoxyribonucleic acid (DNA) base substitutions.

Suitable siRNA compositions that contain two separate oligonucleotides can be chemically linked outside their annealing region by chemical linking groups. Many suitable chemical linking groups are known in the art and can be used. Suitable groups will not block endonuclease activity on the siRNA and will not interfere with the directed destruction of the RNA transcribed from the target gene. Alternatively, the two separate oligonucleotides can be linked by a third oligonucleotide such that a hairpin structure is produced upon annealing of the two oligonucleotides making up the siRNA composition. The hairpin structure will not block endonuclease activity on the siRNA and will not interfere with the directed destruction of the target RNA.

The dsRNA molecules of the disclosed embodiments are added directly, or can be complexed with lipids (e.g., cationic lipids), packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers.

The dsRNA agent can be formulated as a pharmaceutical composition which comprises a pharmacologically effective amount of a dsRNA agent and pharmaceutically acceptable carrier. A pharmacologically or therapeutically effective amount refers to that amount of a dsRNA agent effective to produce the intended pharmacological, therapeutic or preventive result. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20 percent reduction in a measurable parameter associated with a disease or disorder, a therapeutically effective amount of a drug for the treatment of that disease or disorder is the amount necessary to effect at least a 20 percent reduction in that parameter.

The dosages, methods of administration, and times of administration are readily determinable by a person skilled in the art, given the teachings contained herein.

### Dosing

As defined herein, a therapeutically effective amount of a nucleic acid molecule (i.e., an effective dosage, or a therapeutically effective dosage) depends on the nucleic acid selected. For instance, single dose amounts of a dsRNA (or, e.g., a construct(s) encoding for such dsRNA) in the range of approximately 1 pg to up to 10 mg may be administered. In the disclosed embodiments, 1, 10, 30, 100, or 1000 pg, or 10, 30, 100, or 1000 ng, or 10, 30, 100, or 1000 μg, may be administered in one or more areas of the body of a 60 to 120 kg subject.

More particularly for the purposes of treating BCC or isSCC, a dosages administered according to the disclosed embodiments are between about 5 and about 170 μg, between about 10 and about 160 μg, between about 10 and about 130 μg, between about 10 and about 70 μg, between about 10 and about 40 μg, between about 20 and about 50 μg, between about 20 and about 30 μg, between about 30 and about 70 μg, between about 40 and about 80 μg, between about 60 and about 90 μg, between about 50 and about 100 μg, between about 70 and about 100 μg, and between about 80 and about 120 μg, at least once weekly for one to 12 weeks. Dosages and treatment periods will vary. In some embodiments, doses ranging from 60 to 150 μg are administered. Advantageously, the compositions are administered subcutaneously or subdermally in multiple areas where remodeling of the adipose tissue is desired, for example, in submental or adipose tissue. Each dose may be, for example, 60-150m per cm² administered to several areas in a 60 kg to a 120 kg patient. The skilled artisan will recognize that doses greater or lesser than 60-150 μg per cm² may be administered, for example, between 10-300 per cm².

The compositions can be administered from one or more times per day to one or more times per week for the desired length of the treatment from one week up to several months or a year or more; dosages may be administered in some embodiment once every other day. The skilled artisan will appreciate that certain factors may influence the dosage and timing required to effectively treat a subject, including but not limited to the severity of the disease or disorder, previous treatments, the general health and/or age of the subject, and other diseases present. Treatment of a subject with a therapeutically effective amount of a nucleic acid (e.g., dsRNA), protein, polypeptide, or antibody can include a single treatment or, preferably, can include a series of treatments. In a preferred embodiment, one or more doses is administered weekly or semiweekly for a period between one and six to 12 weeks. In another embodiment, one or more doses is administered daily.

In general, based on a per kg body weight unit, a suitable dosage of dsRNA may range between 1 ng to 2 milligrams per kilogram body weight of the recipient per day, week or month, but more likely will be within the narrower range of between about 0.01 to about 20 micrograms per kilogram body weight per day, week or month, or in the range between about 0.001 to about 5 micrograms per kilogram of body weight per day, week or month, or in the range between about 1 to about 500 nanograms per kilogram of body weight per day, week or month, or in the range between about 0.01 to about 10 micrograms per kilogram body weight per day, week or month, or in the range between about 0.10 to about 5 micrograms per kilogram body weight per day, week or month, or in the range between about 0.1 to about 2.5 micrograms per kilogram body weight per day, week or month. A pharmaceutical composition comprising the dsRNA can be administered once daily. However, the therapeutic agent may also be dosed in units containing two, three, four, five, six or more sub-doses administered at appropriate intervals throughout the day, week or month. In that case, the dsRNA contained in each sub-dose must be correspondingly smaller to achieve the total daily, weekly or monthly dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

Depending on the particular target gene sequence and the dose of dsRNA agent material delivered, this process may provide partial or complete loss of function for the target gene. A reduction or loss of expression (either target gene expression or encoded polypeptide expression) in at least 50%, 60%, 70%, 80%, 90%, 95% or 99% or more of targeted cells is exemplary. Inhibition of target gene levels or expression refers to the absence (or observable decrease) in the level of target gene or target gene -encoded protein. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (MA), other immunoassays, and fluorescence activated cell analysis (FACS). Inhibition of target target gene sequence(s) by the dsRNA agents of the disclosed embodiments also can be measured based upon the effect of administration of such dsRNA agents upon development/progression of a target gene associated disease or disorder, e.g., deleterious adipose tissue remodeling due to obesity, over feeding or a metabolic derangement, tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, 10⁷-fold reduction in cancer cell levels could be achieved via administration of the dsRNA agents of the disclosed embodiments to cells, a tissue, or a subject.

The data obtained from the cell culture assays and animal studies (toxicity, therapeutic efficacy) can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the method of the disclosed embodiments, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Administration

Suitably formulated compositions of the disclosed embodiments can be administered by means known in the art such as by parenteral routes, including intravenous, intramuscular, intraperitoneal, subcutaneous, subdermal, transdermal, airway (aerosol), rectal, vaginal and topical (including buccal and sublingual) administration. In some embodiments, the pharmaceutical compositions are administered by intravenous or intra-parenteral infusion or injection. In one embodiment, the composition is administered by injection into the tissue. In another embodiment, the composition is ministered by subcutaneous injection into a mammal. In still another embodiment, the composition is administered topically to the mammal.

A formulation is prepared to be compatible with its intended route of administration. Examples of routes of administration include parenteral, e.g., intravenous, intradermal, subcutaneous, subdermal, oral (e.g., inhalation), transdermal (topical), transmucosal, and rectal administration. Solutions or suspensions used for parenteral, intradermal, subdermal or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The siRNA formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

Further, the siRNA formulations can also be administered by a method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

### Treatments

The presently disclosed embodiments provides for both prophylactic and therapeutic methods of treating a subject at risk of (or susceptible to) a disease, disorder or condition caused or exacerbated, in whole or in part, by TGFβ1 and/or Cox-2 gene expression.

“Treatment”, or “treating” as used herein, is defined as the application or administration of a therapeutic agent (e.g., a dsRNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosed embodiments provides a method for preventing in a subject, a disease or disorder as described above (including, e.g., prevention of the commencement of transforming events within a subject via inhibition of TGFβ1 and Cox-2 expression), by administering to the subject a therapeutic agent (e.g., a dsRNA agent or vector or transgene encoding same). Subjects at risk for the disease can be identified by, for example, one or a combination of diagnostic or prognostic assays as described herein. Administration of a prophylactic agent can occur prior to the detection of, e.g., cancer in a subject, or the manifestation of symptoms characteristic of the disease or disorder, such that the disease or disorder is prevented or, alternatively, delayed in its progression.

The dsRNA molecules (siRNA) can be used in combination with other treatments to treat, inhibit, reduce, or prevent a deleterious adipose remodeling in a subject or organism.

Another aspect of the disclosed embodiments pertains to methods of treating subjects therapeutically, i.e., altering the onset of symptoms of the disease or disorder. These methods can be performed in vitro (e.g., by culturing the cell with the dsRNA agent) or, alternatively, in vivo (e.g., by administering the dsRNA agent to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the disclosed embodiments provides methods for tailoring an individual's prophylactic or therapeutic treatment with either the target TGFβ1 and Cox-2 genes or modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in a selected animal model. For example, a dsRNA agent (or expression vector or transgene encoding same) as described herein can be used in an animal model to determine the efficacy, toxicity, or side effects of treatment with said agent. Alternatively, an agent (e.g., a therapeutic agent) can be used in an animal model to determine the mechanism of action of such an agent.

The disclosed embodiments described and claimed are not to be limited in scope by the specific preferred embodiments referenced herein, since these embodiments are intended as illustrations, not limitations. Any equivalent embodiments are intended to be within the scope of this disclosure, and the embodiments disclosed are not mutually exclusive. Indeed, various modifications to the embodiments, in addition to those shown and described herein will become apparent to those skilled in the art from the foregoing description. Such modifications are also intended to fall within the scope of the appended claims.

The terms and words used in the following description and claims are not limited to conventional definitions but, rather, are used to enable a clear and consistent understanding of the disclosure. Accordingly, it should be apparent to those skilled in the art that the description of various embodiments is provided for illustration purpose only and not for the purpose of limiting the disclosure with respect to the appended claims and their equivalents.

It is to be understood that the singular forms “a,” “an,” and “the” include the plural forms unless the context clearly dictates otherwise, e.g., reference to “a dermatologically active compound” includes reference to one or more such compounds.

Unless otherwise defined herein, all terms used have the same meaning as commonly understood by a person of ordinary skill in the art. Terms used herein should be interpreted as having meanings consistent with their meanings in the context of the relevant art.

As used herein, the terms “comprising,” “comprise” or “comprised,” in reference to defined or described elements of any item, composition, formulation, apparatus, method, process, system, etc., are intended to be inclusive or open ended, and includes those specified elements or their equivalents. Other elements can be included and still fall within the scope or definition of the defined item, composition, etc.

The term “about” or “approximately” means within an acceptable error range for the particular value as viewed by one of ordinary skill in the art; this depends in part on how the value is measured or determined based on the limitations of the measurement system.

“Co-administer” or “co-deliver” refers to the simultaneous administration of two pharmaceutical formulations in the blood or other fluid of an individual using the same or different modes of administration. Pharmaceutical formulations can be concurrently or sequentially administered in the same pharmaceutical carrier or in different ones.

The terms “subject,” “patient,” and “individual” are used interchangeably.
The following examples illustrate certain aspects of the disclosed embodiments and should not be construed as limiting the scope thereof.

### EXAMPLES

### Example 1

Tissue samples used in experiments were from skin excisions of three women (ages 23-26) that had undergone breast reconstruction for treatment of macromastia. All patients provided informed consent. Skin excisions were acquired in sterile condition, cut into size of 2 cm×1.6 cm and kept in 20% FBS DMEM media at 4° C. before use. Human skin hypertrophic scar tissue was obtained from a surgical excision with the informed consent and was trimmed off subcutaneous fat and cut into pieces of 2 cm². Male nude mice (6-8 weeks old) were anesthetized with 10% chloral hydrate and a piece of trimmed hypertrophic scar tissue was implanted under the skin on the mouse back. Scar tissue was fixed to the mouse deep fascia with 4-5 sutures. A same size human skin was grafted to replace the excision by sutures to subcutaneous fascia and surrounding mouse skin. Sterile cotton was positioned on the graft and tightly wrapped, and two weeks later, the stitches were removed. Four weeks after the scar implantation, 20 μg/50 μL/cm³ of HKP (TGFβ1/Cox-2 siRNAs) was injected into the scar on the mouse body. To ensure even drug distributions, injections were performed into 5 areas: 4 quadrants and 1 center. Each drug dose was injected in 5 equal aliquots. Scars were injected three times for 15 days (once every 5 days), and scar size was evaluated before and after treatment. Mice were euthanized, and scar tissue was immediately harvested and homogenized in Trizol solution with Polytrone (Brinkmann Homogenizer Polytron PT 10/35). Total RNA was extracted and RNA level of TGFβ1, Cox-2, α-SMA, Collal, and Col3a1 was analyzed by qRTPCR. The in situ Cell Death Detection Kit from Roche (South San Francisco, CA, USA) was used for detection of apoptotic cells from the STP705 treated scar tissues, following the vendor's instruction. Mean±standard deviation (SD) was used for cell culture results and mean±standard error (SE) was used for in vivo results. The student's t-test was used to determine significance between two groups. P-values less than 0.05 were considered statistically significant. IBM SPSS statistics, version 20, was used for statistical analysis.

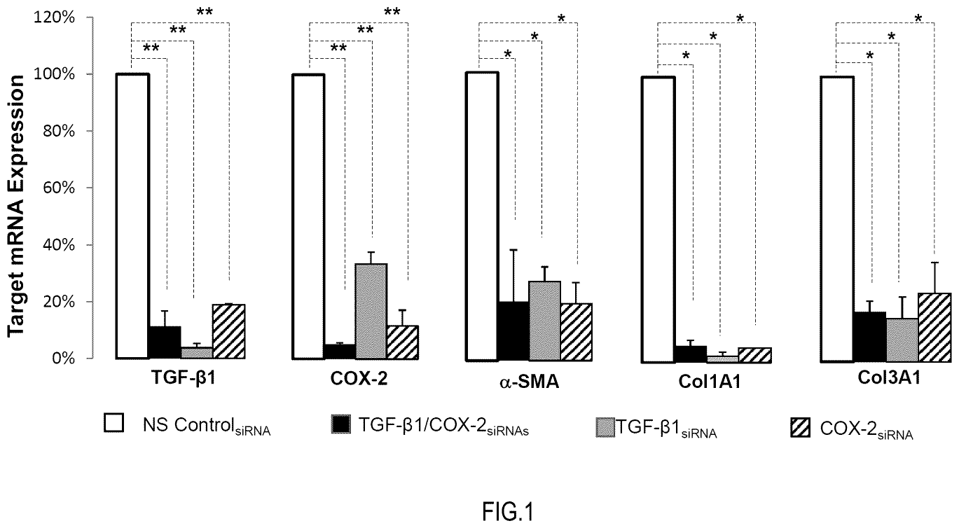

We have demonstrated that administration of these siRNAs in a nanoparticles consisting of HKP (referred to herein as STP705) silences of the targets genes and downstream effects on select targets including alphaSMA, Col1A1 and Col3A1 (Zhou et al., supra). FIG. 1 shows significantly reduced mRNA levels of TGFβ1 and Cox-2, the combination of TGFβ1 and Cox-2, and pro-fibrotic factors such as collagen 1 (Col1A1) from human hypertrophic scar fibroblasts after transfection (5 μg/mL).

### Example 2

Fifty-two subject samples (with 3 samples included from subjects No. 3 and 8) from pre- and post-treatment specimens were chosen based on hematoxylin and eosin review for relative tumor and relative necrosis and tumor content. Samples were stained using optimized immunohistochemistry protocols for TGFβ1 [1:100 (0.59 μg/mL)], Cox-2 [1:100 (5.01 μmg/mL)], NFκB p65 [1:200 (1.04 μmg/mL)], Ki-67 [1:100 (7.04 μg/mL)], β-Catenin [1:200 (0.315 μg/mL)], CD4+ [1:100 (1.43 μg/mL)], and CD8+ [1:50 (9.085 μg/mL)] antibodies sourced from a rabbit (Abcam, Cambridge, England). Staining was done on the Leica BOND III platform using AP Red (Bond Polymer Refine Red Detection kit) (Leica Biosystems, Buffalo Grove, IL) chromogenic secondaries. Biomarker stains were evaluated, providing semiquantitative H-score results, and accounting for percentage of cells with staining intensity based on a scale of 0-3 and the relative percentage of cells at each staining intensity.

The following formula was used to assign an H-score: [1×(% cells 1+)+2×(% cells 2+)+3×(% cells 3+)]. The final, weighted score ranges from 0 to 300.Pretreatment scoring was performed on the tumor and tumor microenvironment. Post-treatment scoring was performed on residual tumor/surface epithelium and adjacent non-tumor scar tissue.

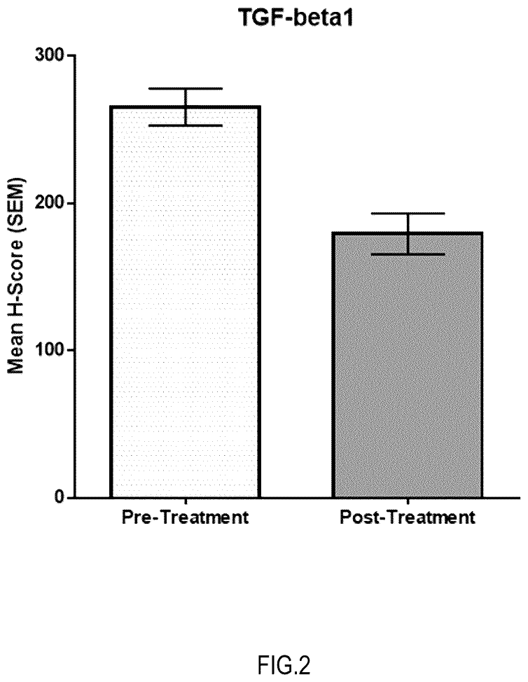

Administration of STP705 to human patients with isSCC resulted in a significant reduction in TGFβ 1 protein expression are shown in FIG. 2. Samples of tissue were obtained (10-30 μg doses of STP705 administered). Protein expression in the matched patient tissue samples were analyzed using immunohistochemistry and semi-quantitatively evaluated by a pathologist.

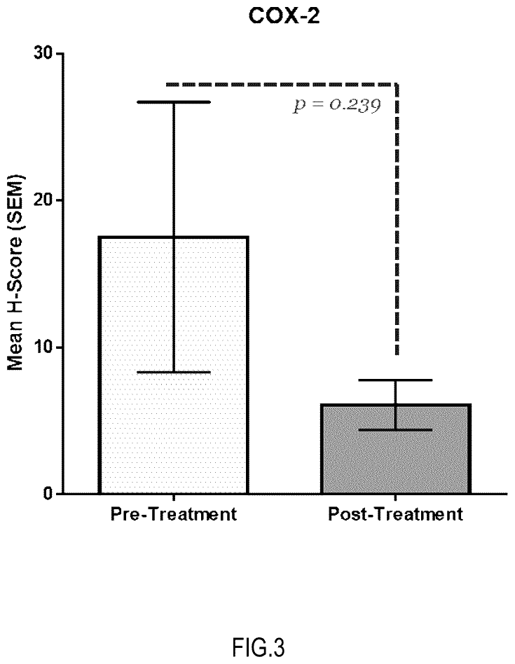

Administration of STP705 to human patients with isSCC resulted in a reduction in COX-2 protein expression as shown in FIG. 3. Samples of tissue were obtained (10-30 μg doses of STP705 administered) and, again, protein expression in the matched patient tissue samples was analyzed using immunohistochemistry and semi-quantitatively evaluated by a pathologist.

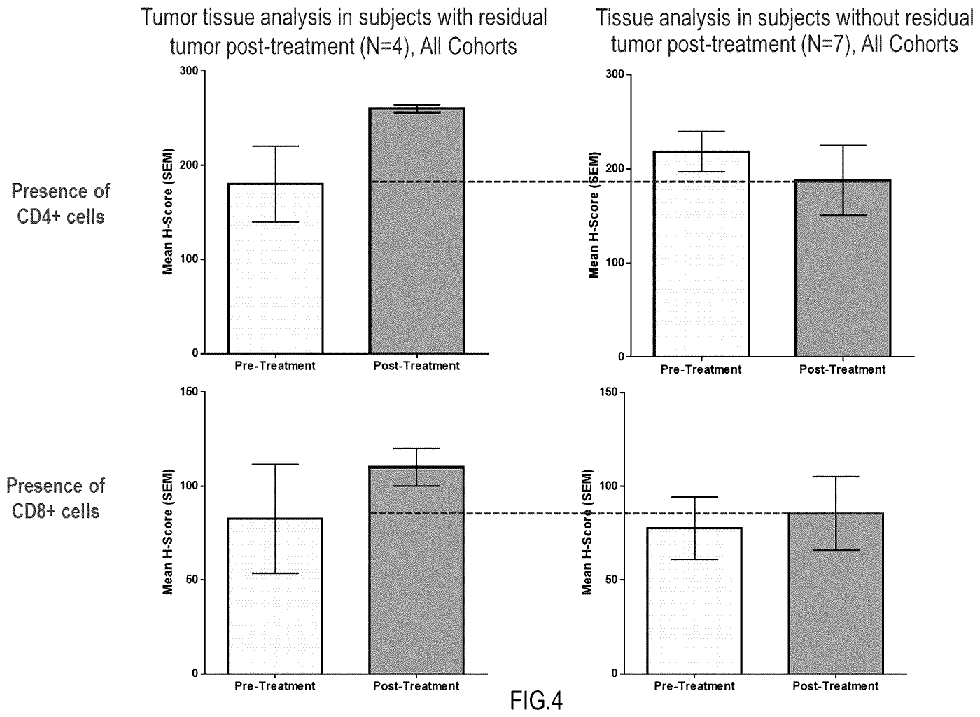

STP705 administration resulted in an increase in T-cell penetration into the tumor sites as shown in FIG. 4. The upper panel (left) shows that patients with residual tumor at the conclusion of the study demonstrated an increased uptake of CD4+ T-cells into the tumor compared to pretreatment conditions. Furthermore (lower panel left) there was also an increase in CD8+ T-cell penetration into the tumor site post treatment with STP705.

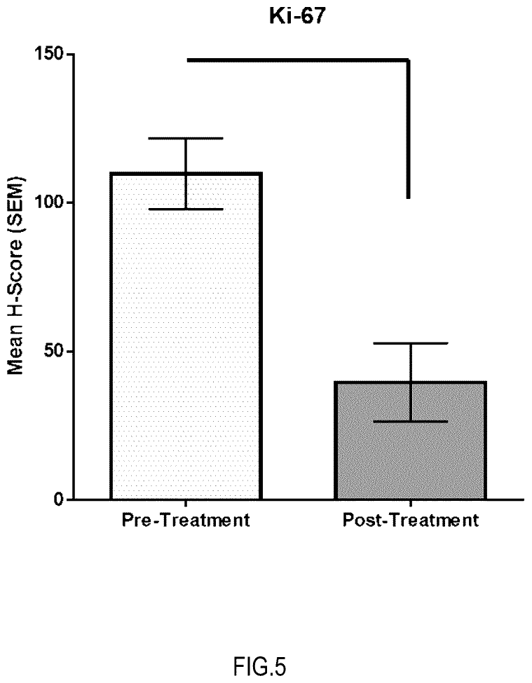

STP705 further inhibits proliferation of cells. Administration of STP705 to human patients with isSCC resulted in a reduction in Ki-67 cell proliferation protein expression as shown in FIG. 5. Samples of tissue were obtained from all comers (10-30 μg doses of STP705 administered). The protein expression in the matched patient tissue samples were analyzed using immunohistochemistry and semi-quantitatively evaluated by a pathologist. Ki67 staining was performed to measure proliferating cells. A dramatic reduction was observed across all-comers post treatment with STP705.

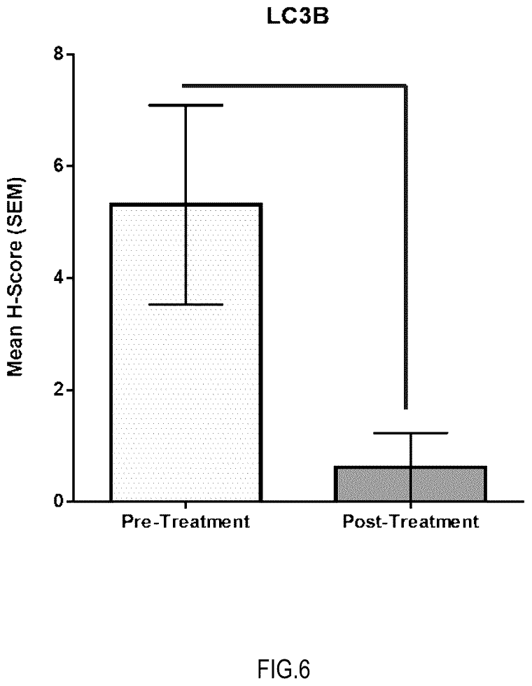

STP705 treatment also inhibits autophagy within the tumor site. Administration of STP705 to human patients with isSCC resulted in reduced expression of LC3B autophagy marker as shown in FIG. 6. Samples of tissue were obtained from all comers (10-30 μg doses of STP705 administered). The protein expression in the matched patient tissue samples were analyzed using immunohistochemistry and semi-quantitatively evaluated by a board-certified MD pathologist. Measuring LC3B as a marker of autophagy we see a dramatic reduction in this marker in all-comers (10-30 μg dose) compared with pretreatment levels (p<0.031, treated vs. pre-treatment).

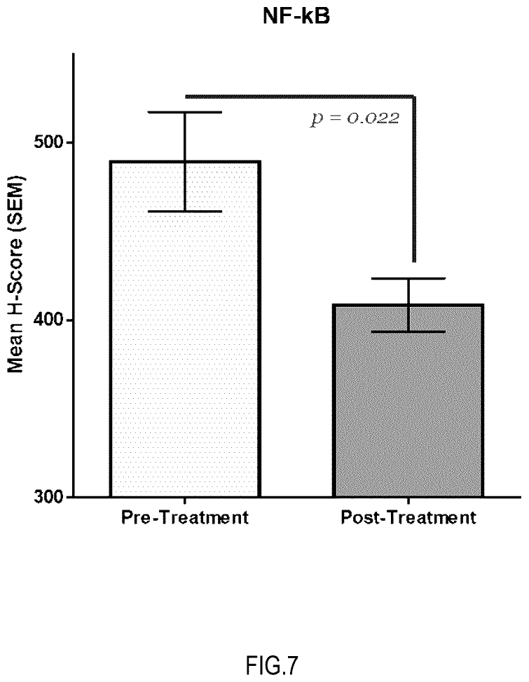

The effect of STP705 on NFkB levels. Administration of STP705 to human patients with isSCC resulted in reduced expression of NF-kB protein as shown in FIG. 7. Samples of tissue were obtained from all comers (10-30 ug doses of STP705 administered). The protein expression in the matched patient tissue samples were analyzed using immunohistochemistry and semi-quantitatively evaluated by a board-certified MD pathologist. Treatment with STP705 diminishes the amount of NFkB present within the tumor site. p=0.022 between treated and untreated samples.

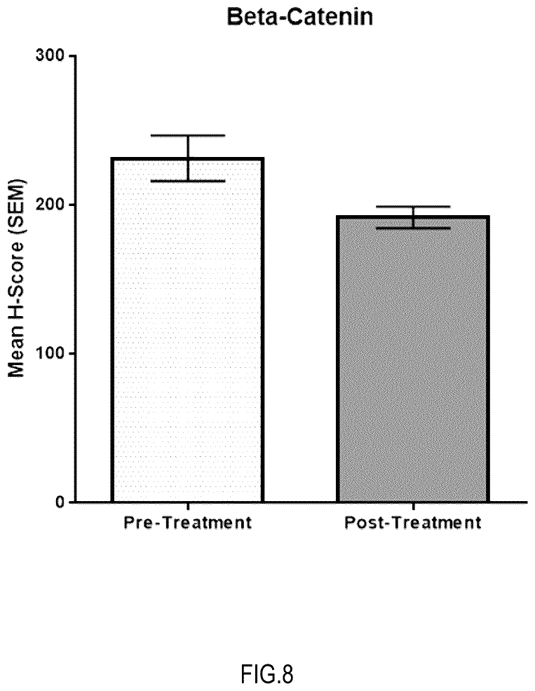

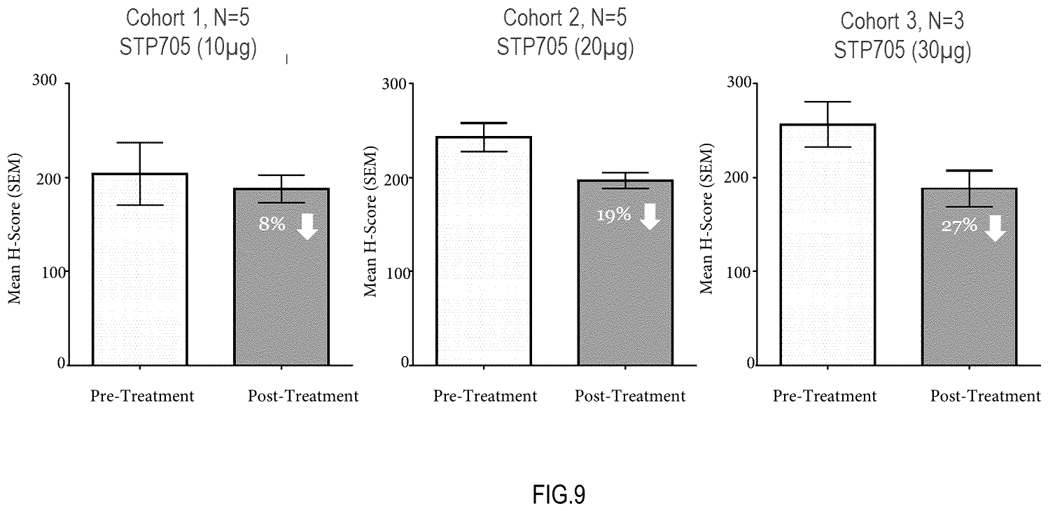

Effect of STP705 on β-Catenin levels within the tumor. Administration of STP705 to human patients with isSCC resulted in reduced expression of β-Catenin as shown in FIG. 8. Samples of tissue were obtained from all comers (10-30 μg doses of STP705). The protein expression in the matched patient tissue samples were analyzed using immunohistochemistry and semi-quantitatively evaluated by a pathologist. Post treatment with STP705 showed a reduction in β-catenin level within the tumor region of all-comers (10-30 μg dose). This was dose-dependent as shown in FIG. 9.

We have also shown in isSCC patients that IT administration of STP705 resulted in a clearance of the tumor cells from the lesion in a dose dependent manner and resulted in little/no scarring on the patients' skin. This is especially important since many of these lesions occur in exposed regions like the face/neck where scarring from current treatment regimens (Surgery or Curettage and Electro desiccation) is commonplace. These data (not shown) demonstrate that administration of siRNAs against TGFβ1 and Cox-2 in a single nanoparticle delivery system to obtain co-delivery of both siRNAs to the same cells at the same time shows surprising and powerful activity against in situ Squamous Cell Carcinoma (isSCC). The same formulation and administration methods can be used to treat basal cell carcinoma.

### Example 3

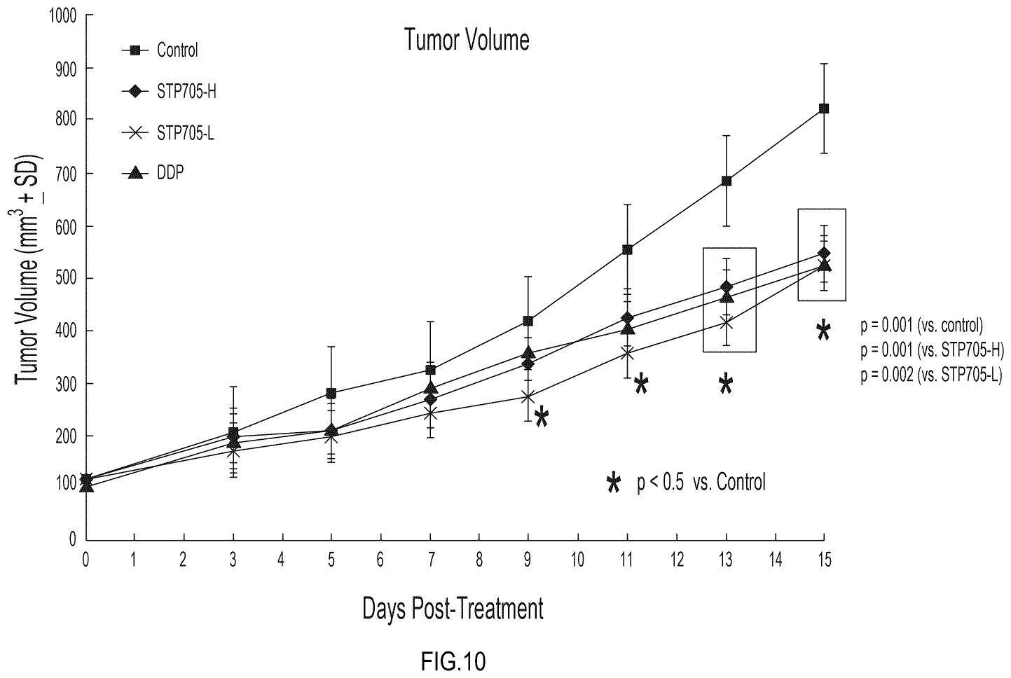

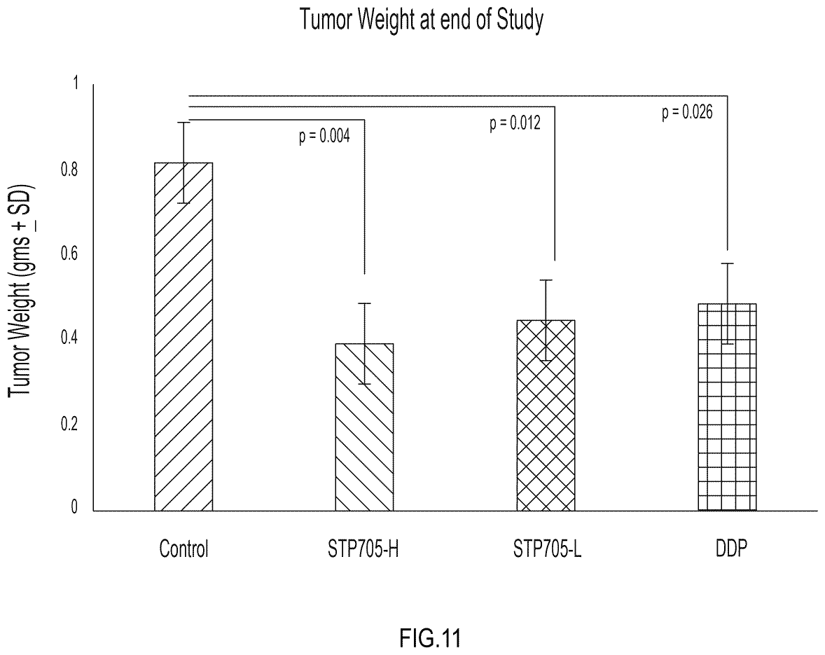

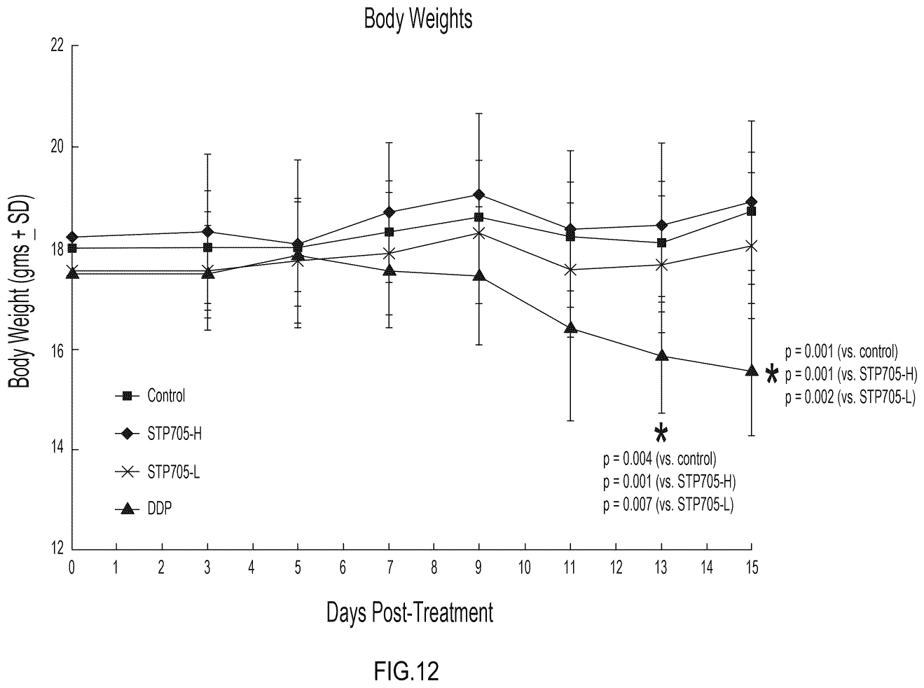

A xenograft mouse tumor model and A431 human squamous cell carcinoma cell lines were used in a pre-clinical proof of concept evaluation. Mice were dosed twice weekly over 15 days with high (40 μg) and low (20 μg) dose STP750 or Cisplatin (DPP). FIG. 10 shows the significant (p<0.05) attenuation in the increase in tumor size over time with administration of STP705 in high and low doses. FIG. 11 shows significantly reduced tumor weights. FIG. 12 shows maintenance of body weight with high and low dose STP705 versus significant loss of body weight in patients following administration of DPP.

### Example 4

In vivo study to evaluate the safety, tolerability and efficacy of escalating doses of STP705 administered as localized injection in patients with Basal Cell Carcinoma (BCC).

Methods: Clinical Protocol: A Phase 2, open label, dose escalation study is designed to evaluate the safety, tolerability and efficacy of various doses of STP705 administered as localized injection in patients with basal cell carcinoma (BCC), and no evidence of isSCC or other non-BCC tumor in a biopsy specimen. Study Design: Fifteen adult subjects (5 per cohort) were assigned to receive the treatment if eligible, with dosing regimen as follows: Cohort A: STP705 30 μg dose, intradermal injection once a week for up to 6 weeks; Cohort B: STP705 60 μg dose, intradermal injection, given once a week for up to 6 weeks; Cohort C: STP705 90 μg dose, intradermal injection, given once a week for up to 6 weeks.

Primary Endpoints: The proportion of participants with histological clearance of treated basal cell carcinoma lesion at the end of treatment (6 weeks). Histological clearance (HC) will be defined as the absence of detectable evidence of BCC tumor cell nests as determined by central pathology review. Secondary Endpoints: Determination of the safe and effective recommended dose of STP705 for the treatment of BCC; analysis of biomarkers common to BCC formation pathway including TGFβ1 and Cox-2. Complete information for this BCC clinical trial due to end in 2022 is available at: https://clinicaltrials.gov/ct2/show/NCT04669808?term=sirnaomics&draw=2&rank=4,

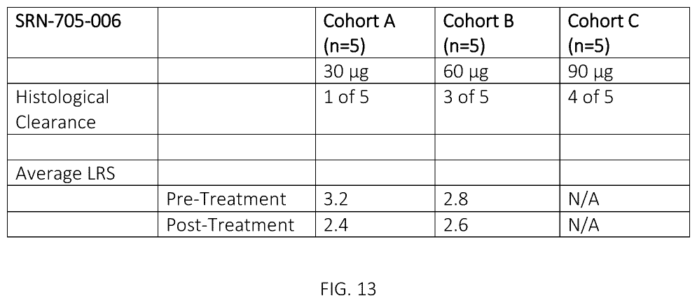

FIG. 13 shows a table with pre-and post-treatment average local response score (LRS) for Cohorts A (30 μmg dose), and B (60 μmg dose); histological clearance data is available for Cohorts A, B and C (90 μg dose) at this time. Available data indicates that even at the lower doses, BCC tumor growth is attenuated or inhibited. These early data indicate a dose response for histologic clearance of BCC tumor tissue. This study is ongoing, and a new Cohort (D) (n=4, dose: 120 μmg) recently has been added to the study but only some Cohort C data and no data from Cohort D are yet available. Results of a random sample of subjects who achieved Complete Response (CR=complete histological clearance of tumor cells) revealed improved LRSs post-treatment compared to pretreatment. LRSs are the most common adverse effects witnessed during clinical studies of topical or locally-injected therapeutics. Improved LSRs suggest fewer local adverse events and improved appearance of the skin in the treated area.

We claim:

1. A method of treating in situ squamous cell carcinoma (isSCC) or basal cell carcinoma (BCC), comprising administering to a patient suffering from isSCC and/or BCC an effective amount of a nanoparticle formulation comprising at least one siRNA that inhibits the activity of TGFβ 1 and at least one siRNA that inhibits Cox-2.

2. The method of claim 1 where said nanoparticle formulation comprises HKP and/or HKP(+H).

3. The method of claim 1 wherein said formulation is administered through intra-tumoral injection.

4. The method of claim 1 wherein said formulation is administered to the tumor through intravenous (systemic) administration.

5. The method of claim 1, wherein the formulation product is administered together with an immune checkpoint inhibitor.

6. The method of claim 5 wherein said immune checkpoint inhibitor is an antibody or other agent that binds or inhibits a checkpoint protein selected from the group consisting of PD-1, PDL1, Lag3, Tim3, and CTLA-4/B7.

7. The method of claim 6 wherein said immune checkpoint inhibitor is a PD-1 inhibitor.

8. The method of claim 7, wherein said PD-1 inhibitor is selected from the group consisting of Pembrolizumab (Keytruda), Nivolumab (Opdivo), and Cemiplimab (Libtayo).

9. The method of claim 6 wherein said immune checkpoint inhibitor is a PD-L1 inhibitor.

10. The method of claim 9 wherein said PD-L1 inhibitor is selected from the group consisting of Atezolizumab (Tecentriq), Avelumab (Bavencio), and Durvalumab (Imfinzi).

11. The method of claim 6 wherein said immune checkpoint inhibitor is a CTLA-4 inhibitor.

12. The method of claim 11 wherein said CTLA-4 inhibitor is Ipilimumab (Yervoy).

13. The method of claim 6 wherein said immune checkpoint inhibitor is a lymphocyte activation gene-3 (LAG-3) inhibitor.

14. The method of claim 13, wherein said LAG-3 inhibitor is BMS-986016.

15. The method of claim 6, wherein said immune checkpoint inhibitor targets T cell immunoglobulin and mucin-domain containing-3 (TIM-3), T cell immunoglobulin and ITIM domain (TIGIT), V-domain Ig suppressor of T cell activation (VISTA), B7 homolog 3 protein (B7-H3) or B and T cell lymphocyte attenuator (BTLA)).

16. A method of treating basal cell carcinoma (BCC), comprising administering to a patient suffering from BCC an effective amount of a pharmaceutical composition comprising at least one siRNA that inhibits the activity of TGFβ1 and at least one siRNA that inhibits Cox-2, wherein the composition is administered to the patient in a dosage of between about 5 and 170 μg, at least once weekly for between about 1 and 12 weeks, and wherein tumor growth of the BCC in the patient is attenuated or inhibited.

17. The method of claim 16, wherein the pharmaceutical composition is administered to the patient in a dosage selected from the group consisting of between about 10 and about 160 μg, between about 10 and about 130 μg, between about 10 and about 70 μg, between about 10 and about 40 μg, between about 20 and about 50 μg, between about 20 and about 30 μg, between about 30 and about 70 μg, between about 40 and about 80 μg, between about 60 and about 90 μg, between about 50 and about 100 μg, and between about 70 and about 100 μg, and between about 80 and about 120 μg, at least once weekly for one to 12 weeks.

18. The method of claim 16, wherein a local skin response (LSR) in the patient is reduced post-treatment.

19. The method of claim 16, wherein a histological clearance of BCC in the patient is dose-dependent.


SEQUENCE LISTING (104 sequences)

SEQ ID NO: 1 - RNA, 25 nt, synthetic construct
  misc_feature 1..25: note = Description of Artificial Sequence: Synthetic oligonucleotide
  source 1..25: mol_type = other RNA
ggtctggtgcctggtctgatgatgt

SEQ ID NO: 2 - RNA, 25 nt, Homo sapiens
  source 1..25: mol_type = genomic RNA
ggtctggtgcctggtctgatgatgt

SEQ ID NO: 3 - RNA, 25 nt, Mus sp.
  source 1..25: mol_type = genomic RNA
ggtctggtgcctggtctgatgatgt

SEQ ID NO: 4 - RNA, 25 nt, Simian sp.
  misc_feature 1..25: note = Description of Unknown: Monkey TGFBeta1 sequence
  source 1..25: mol_type = genomic RNA
ggtctggtgcctggtctgatgatgt

SEQ ID NO: 5 - RNA, 25 nt, Sus sp.
  misc_feature 1..25: note = Description of Unknown: Pig TGFBeta1 sequence
  source 1..25: mol_type = genomic RNA
ggtctggtgcctggtctgatgatgt

SEQ ID NO: 6 - RNA, 25 nt, synthetic construct
  misc_feature 1..25: note = Description of Artificial Sequence: Synthetic oligonucleotide
  source 1..25: mol_type = other RNA
cccaagggctaccatgccaacttct

SEQ ID NO: 7 - RNA, 25 nt, synthetic construct
  misc_feature 1..25: note = Description of Artificial Sequence: Synthetic oligonucleotide
  source 1..25: mol_type = other RNA
agaagttggcatggtagcccttggg

SEQ ID NO: 8 - RNA, 25 nt, synthetic construct
  misc_feature 1..25: note = Description of Artificial Sequence: Synthetic oligonucleotide
  source 1..25: mol_type = other RNA
ggtctggtgcctggtctgatgatgt

SEQ ID NO: 9 - RNA, 25 nt, synthetic construct
  misc_feature 1..25: note = Description of Artificial Sequence: Synthetic oligonucleotide
  source 1..25: mol_type = other RNA
acatcatcagaccaggcaccagacc

SEQ ID NO: 10 - RNA, 25 nt, synthetic construct
  misc_feature 1..25: note = Description of Artificial Sequence: Synthetic oligonucleotide
  source 1..25: mol_type = other RNA
ggtggctggaacagccagatgtgtt

SEQ ID NO: 11 - RNA, 25 nt, synthetic construct
  misc_feature 1..25: note = Description of Artificial Sequence: Synthetic oligonucleotide
  source 1..25: mol_type = other RNA
aacacatctggctgttccagccacc

SEQ ID NO: 12 - RNA, 25 nt, synthetic construct
  misc_feature 1..25: note = Description of Artificial Sequence: Synthetic oligonucleotide
  source 1..25: mol_type = other RNA
gaggagccttcaggattacaagatt

SEQ ID NO: 13 - RNA, 25 nt, synthetic construct
  misc_feature 1..25: note = Description of Artificial Sequence: Synthetic oligonucleotide
  source 1..25: mol_type = other RNA
aatcttgtaatcctgaaggctcctc

SEQ ID NO: 14 - RNA, 25 nt, synthetic construct
  misc_feature 1..25: note = Description of Artificial Sequence: Synthetic oligonucleotide
  source 1..25: mol_type = other RNA
gctgaccctgaagttcatctgcatt

SEQ ID NO: 15 - RNA, 25 nt, synthetic construct
  misc_feature 1..25: note = Description of Artificial Sequence: Synthetic oligonucleotide
  source 1..25: mol_type = other RNA
aatgcagatgaacttcagggtcagc

SEQ ID NO: 16 - RNA, 25 nt, synthetic construct
  misc_feature 1..25: note = Description of Artificial Sequence: Synthetic oligonucleotide
  source 1..25: mol_type = other RNA
ggatccacgagcccaagggctacca

SEQ ID NO: 17 - RNA, 25 nt, synthetic construct
  misc_feature 1..25: note = Description of Artificial Sequence: Synthetic oligonucleotide
  source 1..25: mol_type = other RNA
tggtagcccttgggctcgtggatcc

SEQ ID NO: 18 - RNA, 25 nt, synthetic construct
  misc_feature 1..25: note = Description of Artificial Sequence: Synthetic oligonucleotide
  source 1..25: mol_type = other RNA
cccaagggctaccatgccaacttct

SEQ ID NO: 19 - RNA, 25 nt, synthetic construct
  misc_feature 1..25: note = Description of Artificial Sequence: Synthetic oligonucleotide
  source 1..25: mol_type = other RNA
agaagttggcatggtagcccttggg

SEQ ID NO: 20 - RNA, 25 nt, synthetic construct
  misc_feature 1..25: note = Description of Artificial Sequence: Synthetic oligonucleotide
  source 1..25: mol_type = other RNA
gagcccaagggctaccatgccaact

SEQ ID NO: 21 - RNA, 25 nt, synthetic construct
  misc_feature 1..25: note = Description of Artificial Sequence: Synthetic oligonucleotide
  source 1..25: mol_type = other RNA
agttggcatggtagcccttgggctc

SEQ ID NO: 22 - RNA, 25 nt, synthetic construct
  misc_feature 1..25: note = Description of Artificial Sequence: Synthetic oligonucleotide
  source 1..25: mol_type = other RNA
gatccacgagcccaagggctaccat

SEQ ID NO: 23 - RNA, 25 nt, synthetic construct
  misc_feature 1..25: note = Description of Artificial Sequence: Synthetic oligonucleotide
  source 1..25: mol_type = other RNA
atggtagcccttgggctcgtggatc

SEQ ID NO: 24 - RNA, 25 nt, synthetic construct
  misc_feature 1..25: note = Description of Artificial Sequence: Synthetic oligonucleotide
  source 1..25: mol_type = other RNA
cacgagcccaagggctaccatgcca

SEQ ID NO: 25 - RNA, 25 nt, synthetic construct
  misc_feature 1..25: note = Description of Artificial Sequence: Synthetic oligonucleotide
  source 1..25: mol_type = other RNA
tggcatggtagcccttgggctcgtg

SEQ ID NO: 26 - RNA, 25 nt, synthetic construct
  misc_feature 1..25: note = Description of Artificial Sequence: Synthetic oligonucleotide
  source 1..25: mol_type = other RNA
gaggtcacccgcgtgctaatggtgg

SEQ ID NO: 27 - RNA, 25 nt, synthetic construct
  misc_feature 1..25: note = Description of Artificial Sequence: Synthetic oligonucleotide
  source 1..25: mol_type = other RNA
ccaccattagcacgcgggtgacctc

SEQ ID NO: 28 - RNA, 25 nt, synthetic construct
  misc_feature 1..25: note = Description of Artificial Sequence: Synthetic oligonucleotide
  source 1..25: mol_type = other RNA
gtacaacagcacccgcgaccgggtg

SEQ ID NO: 29 - RNA, 25 nt, synthetic construct
  misc_feature 1..25: note = Description of Artificial Sequence: Synthetic oligonucleotide
  source 1..25: mol_type = other RNA
cacccggtcgcgggtgctgttgtac

SEQ ID NO: 30 - RNA, 25 nt, synthetic construct
  misc_feature 1..25: note = Description of Artificial Sequence: Synthetic oligonucleotide
  source 1..25: mol_type = other RNA
gtggatccacgagcccaagggctac

SEQ ID NO: 31 - RNA, 25 nt, synthetic construct
  misc_feature 1..25: note = Description of Artificial Sequence: Synthetic oligonucleotide
  source 1..25: mol_type = other RNA
gtagcccttgggctcgtggatccac

SEQ ID NO: 32 - RNA, 25 nt, synthetic construct
  misc_feature 1..25: note = Description of Artificial Sequence: Synthetic oligonucleotide
  source 1..25: mol_type = other RNA
ggtctggtgcctggtctgatgatgt

SEQ ID NO: 33 - RNA, 25 nt, synthetic construct
  misc_feature 1..25: note = Description of Artificial Sequence: Synthetic oligonucleotide
  source 1..25: mol_type = other RNA
acatcatcagaccaggcaccagacc

SEQ ID NO: 34 - RNA, 25 nt, synthetic construct
  misc_feature 1..25: note = Description of Artificial Sequence: Synthetic oligonucleotide
  source 1..25: mol_type = other RNA
gagcaccattctccttgaaaggact

SEQ ID NO: 35 - RNA, 25 nt, synthetic construct
  misc_feature 1..25: note = Description of Artificial Sequence: Synthetic oligonucleotide
  source 1..25: mol_type = other RNA
agtcctttcaaggagaatggtgctc

SEQ ID NO: 36 - RNA, 25 nt, synthetic construct
  misc_feature 1..25: note = Description of Artificial Sequence: Synthetic oligonucleotide
  source 1..25: mol_type = other RNA
cctcaattcagtctctcatctgcaa

SEQ ID NO: 37 - RNA, 25 nt, synthetic construct
  misc_feature 1..25: note = Description of Artificial Sequence: Synthetic oligonucleotide
  source 1..25: mol_type = other RNA
ttgcagatgagagactgaattgagg

SEQ ID NO: 38 - RNA, 25 nt, synthetic construct
  misc_feature 1..25: note = Description of Artificial Sequence: Synthetic oligonucleotide
  source 1..25: mol_type = other RNA
gatgtttgcattctttgcccagcac

SEQ ID NO: 39 - RNA, 25 nt, synthetic construct
  misc_feature 1..25: note = Description of Artificial Sequence: Synthetic oligonucleotide
  source 1..25: mol_type = other RNA
gtgctgggcaaagaatgcaaacatc

SEQ ID NO: 40 - RNA, 25 nt, synthetic construct
  misc_feature 1..25: note = Description of Artificial Sequence: Synthetic oligonucleotide
  source 1..25: mol_type = other RNA
gtctttggtctggtgcctggtctga

SEQ ID NO: 41 - RNA, 25 nt, synthetic construct
  misc_feature 1..25: note = Description of Artificial Sequence: Synthetic oligonucleotide
  source 1..25: mol_type = other RNA
tcagaccaggcaccagaccaaagac

SEQ ID NO: 42 - RNA, 25 nt, synthetic construct
  misc_feature 1..25: note = Description of Artificial Sequence: Synthetic oligonucleotide
  source 1..25: mol_type = other RNA
gtgcctggtctgatgatgtatgcca

SEQ ID NO: 43 - RNA, 25 nt, synthetic construct
  misc_feature 1..25: note = Description of Artificial Sequence: Synthetic oligonucleotide
  source 1..25: mol_type = other RNA
tggcatacatcatcagaccaggcac

SEQ ID NO: 44 - RNA, 25 nt, synthetic construct
  misc_feature 1..25: note = Description of Artificial Sequence: Synthetic oligonucleotide
  source 1..25: mol_type = other RNA
caccattctccttgaaaggacttat

SEQ ID NO: 45 - RNA, 25 nt, synthetic construct
  misc_feature 1..25: note = Description of Artificial Sequence: Synthetic oligonucleotide
  source 1..25: mol_type = other RNA
ataagtcctttcaaggagaatggtg

SEQ ID NO: 46 - RNA, 25 nt, synthetic construct
  misc_feature 1..25: note = Description of Artificial Sequence: Synthetic oligonucleotide
  source 1..25: mol_type = other RNA
caattcagtctctcatctgcaataa

SEQ ID NO: 47 - RNA, 25 nt, synthetic construct
  misc_feature 1..25: note = Description of Artificial Sequence: Synthetic oligonucleotide
  source 1..25: mol_type = other RNA
ttattgcagatgagagactgaattg

SEQ ID NO: 48 - RNA, 25 nt, synthetic construct
  misc_feature 1..25: note = Description of Artificial Sequence: Synthetic oligonucleotide
  source 1..25: mol_type = other RNA
ggtggctggaacagccagatgtgtt

SEQ ID NO: 49 - RNA, 25 nt, synthetic construct
  misc_feature 1..25: note = Description of Artificial Sequence: Synthetic oligonucleotide
  source 1..25: mol_type = other RNA
aacacatctggctgttccagccacc

SEQ ID NO: 50 - RNA, 25 nt, synthetic construct
  misc_feature 1..25: note = Description of Artificial Sequence: Synthetic oligonucleotide
  source 1..25: mol_type = other RNA
gctggaacagccagatgtgttgcca

SEQ ID NO: 51 - RNA, 25 nt, synthetic construct
  misc_feature 1..25: note = Description of Artificial Sequence: Synthetic oligonucleotide
  source 1..25: mol_type = other RNA
tggcaacacatctggctgttccagc

SEQ ID NO: 52 - RNA, 25 nt, synthetic construct
  misc_feature 1..25: note = Description of Artificial Sequence: Synthetic oligonucleotide
  source 1..25: mol_type = other RNA
cgccagattaccatctggtttcaga

SEQ ID NO: 53 - RNA, 25 nt, synthetic construct
  misc_feature 1..25: note = Description of Artificial Sequence: Synthetic oligonucleotide
  source 1..25: mol_type = other RNA
tctgaaaccagatggtaatctggcg

SEQ ID NO: 54 - RNA, 25 nt, synthetic construct
  misc_feature 1..25: note = Description of Artificial Sequence: Synthetic oligonucleotide
  source 1..25: mol_type = other RNA
ggagcccggcaattatgccaccttg

SEQ ID NO: 55 - RNA, 25 nt, synthetic construct
  misc_feature 1..25: note = Description of Artificial Sequence: Synthetic oligonucleotide
  source 1..25: mol_type = other RNA
caaggtggcataattgccgggctcc

SEQ ID NO: 56 - RNA, 25 nt, synthetic construct
  misc_feature 1..25: note = Description of Artificial Sequence: Synthetic oligonucleotide
  source 1..25: mol_type = other RNA
caaggatatcgaaggcttgctggga

SEQ ID NO: 57 - RNA, 25 nt, synthetic construct
  misc_feature 1..25: note = Description of Artificial Sequence: Synthetic oligonucleotide
  source 1..25: mol_type = other RNA
tcccagcaagccttcgatatccttg

SEQ ID NO: 58 - RNA, 25 nt, synthetic construct
  misc_feature 1..25: note = Description of Artificial Sequence: Synthetic oligonucleotide
  source 1..25: mol_type = other RNA
ggacaagaggcgcaagatctcggca

SEQ ID NO: 59 - RNA, 25 nt, synthetic construct
  misc_feature 1..25: note = Description of Artificial Sequence: Synthetic oligonucleotide
  source 1..25: mol_type = other RNA
tgccgagatcttgcgcctcttgtcc

SEQ ID NO: 60 - RNA, 25 nt, synthetic construct
  misc_feature 1..25: note = Description of Artificial Sequence: Synthetic oligonucleotide
  source 1..25: mol_type = other RNA
gcaagatctcggcagccaccagcct

SEQ ID NO: 61 - RNA, 25 nt, synthetic construct
  misc_feature 1..25: note = Description of Artificial Sequence: Synthetic oligonucleotide
  source 1..25: mol_type = other RNA
aggctggtggctgccgagatcttgc

SEQ ID NO: 62 - RNA, 25 nt, synthetic construct
  misc_feature 1..25: note = Description of Artificial Sequence: Synthetic oligonucleotide
  source 1..25: mol_type = other RNA
ccatctggtttcagaaccgccgggt

SEQ ID NO: 63 - RNA, 25 nt, synthetic construct
  misc_feature 1..25: note = Description of Artificial Sequence: Synthetic oligonucleotide
  source 1..25: mol_type = other RNA
acccggcggttctgaaaccagatgg

SEQ ID NO: 64 - RNA, 25 nt, synthetic construct
  misc_feature 1..25: note = Description of Artificial Sequence: Synthetic oligonucleotide
  source 1..25: mol_type = other RNA
tggtagcccttgggctcgtggatcc

SEQ ID NO: 65 - RNA, 25 nt, synthetic construct
  misc_feature 1..25: note = Description of Artificial Sequence: Synthetic oligonucleotide
  source 1..25: mol_type = other RNA
agaagttggcatggtagcccttggg

SEQ ID NO: 66 - RNA, 25 nt, synthetic construct
  misc_feature 1..25: note = Description of Artificial Sequence: Synthetic oligonucleotide
  source 1..25: mol_type = other RNA
ttgcagatgagagactgaattgagg

SEQ ID NO: 67 - RNA, 25 nt, synthetic construct
  misc_feature 1..25: note = Description of Artificial Sequence: Synthetic oligonucleotide
  source 1..25: mol_type = other RNA
atggtagcccttgggctcgtggatc

SEQ ID NO: 68 - RNA, 25 nt, synthetic construct
  misc_feature 1..25: note = Description of Artificial Sequence: Synthetic oligonucleotide
  source 1..25: mol_type = other RNA
tggcatggtagcccttgggctcgtg

SEQ ID NO: 69 - RNA, 25 nt, synthetic construct
  misc_feature 1..25: note = Description of Artificial Sequence: Synthetic oligonucleotide
  source 1..25: mol_type = other RNA
agaagttggcatggtagcccttggg

SEQ ID NO: 70 - RNA, 25 nt, synthetic construct
  misc_feature 1..25: note = Description of Artificial Sequence: Synthetic oligonucleotide
  source 1..25: mol_type = other RNA
cacccggtcgcgggtgctgttgtac

SEQ ID NO: 71 - RNA, 25 nt, synthetic construct
  misc_feature 1..25: note = Description of Artificial Sequence: Synthetic oligonucleotide
  source 1..25: mol_type = other RNA
gtagcccttgggctcgtggatccac

SEQ ID NO: 72 - RNA, 25 nt, synthetic construct
  misc_feature 1..25: note = Description of Artificial Sequence: Synthetic oligonucleotide
  source 1..25: mol_type = other RNA
acatcatcagaccaggcaccagacc

SEQ ID NO: 73 - RNA, 25 nt, synthetic construct
  misc_feature 1..25: note = Description of Artificial Sequence: Synthetic oligonucleotide
  source 1..25: mol_type = other RNA
agtcctttcaaggagaatggtgctc

SEQ ID NO: 74 - RNA, 25 nt, synthetic construct
  misc_feature 1..25: note = Description of Artificial Sequence: Synthetic oligonucleotide
  source 1..25: mol_type = other RNA
ttgcagatgagagactgaattgagg

SEQ ID NO: 75 - RNA, 25 nt, synthetic construct
  misc_feature 1..25: note = Description of Artificial Sequence: Synthetic oligonucleotide
  source 1..25: mol_type = other RNA
gtgctgggcaaagaatgcaaacatc

SEQ ID NO: 76 - RNA, 25 nt, synthetic construct
  misc_feature 1..25: note = Description of Artificial Sequence: Synthetic oligonucleotide
  source 1..25: mol_type = other RNA
tcagaccaggcaccagaccaaagac

SEQ ID NO: 77 - RNA, 25 nt, synthetic construct
  misc_feature 1..25: note = Description of Artificial Sequence: Synthetic oligonucleotide
  source 1..25: mol_type = other RNA
tggcatacatcatcagaccaggcac

SEQ ID NO: 78 - RNA, 25 nt, synthetic construct
  misc_feature 1..25: note = Description of Artificial Sequence: Synthetic oligonucleotide
  source 1..25: mol_type = other RNA
ataagtcctttcaaggagaatggtg

SEQ ID NO: 79 - RNA, 25 nt, synthetic construct
  misc_feature 1..25: note = Description of Artificial Sequence: Synthetic oligonucleotide
  source 1..25: mol_type = other RNA
ttattgcagatgagagactgaattg

SEQ ID NO: 80 - RNA, 25 nt, synthetic construct
  misc_feature 1..25: note = Description of Artificial Sequence: Synthetic oligonucleotide
  source 1..25: mol_type = other RNA
cccaagggctaccatgccaacttct

SEQ ID NO: 81 - RNA, 25 nt, Homo sapiens
  source 1..25: mol_type = genomic RNA
cccaagggctaccatgccaacttct

SEQ ID NO: 82 - RNA, 25 nt, Mus sp.
  source 1..25: mol_type = genomic RNA
cccaagggctaccatgccaacttct

SEQ ID NO: 83 - RNA, 25 nt, simian sp.
  misc_feature 1..25: note = Description of Unknown: Monkey Cox-2 sequence
  source 1..25: mol_type = genomic RNA
cccaagggctaccatgccaacttct

SEQ ID NO: 84 - RNA, 25 nt, sus sp.
  misc_feature 1..25: note = Description of Unknown: Pig Cox-2 sequence
  source 1..25: mol_type = genomic RNA
cccaagggctaccatgccaatttct

SEQ ID NO: 85 - RNA, 25 nt, synthetic construct
  misc_feature 1..25: note = Description of Artificial Sequence: Synthetic oligonucleotide
  source 1..25: mol_type = other RNA
ggatccacgagcccaagggctacca

SEQ ID NO: 86 - RNA, 25 nt, synthetic construct
  misc_feature 1..25: note = Description of Artificial Sequence: Synthetic oligonucleotide
  source 1..25: mol_type = other RNA
cccaagggctaccatgccaacttct

SEQ ID NO: 87 - RNA, 25 nt, synthetic construct
  misc_feature 1..25: note = Description of Artificial Sequence: Synthetic oligonucleotide
  source 1..25: mol_type = other RNA
cctcaattcagtctctcatctgcaa

SEQ ID NO: 88 - RNA, 25 nt, synthetic construct
  misc_feature 1..25: note = Description of Artificial Sequence: Synthetic oligonucleotide
  source 1..25: mol_type = other RNA
gatccacgagcccaagggctaccat

SEQ ID NO: 89 - RNA, 25 nt, synthetic construct
  misc_feature 1..25: note = Description of Artificial Sequence: Synthetic oligonucleotide
  source 1..25: mol_type = other RNA
cacgagcccaagggctaccatgcca

SEQ ID NO: 90 - RNA, 25 nt, synthetic construct
  misc_feature 1..25: note = Description of Artificial Sequence: Synthetic oligonucleotide
  source 1..25: mol_type = other RNA
gaggtcacccgcgtgctaatggtgg

SEQ ID NO: 91 - RNA, 25 nt, synthetic construct
  misc_feature 1..25: note = Description of Artificial Sequence: Synthetic oligonucleotide
  source 1..25: mol_type = other RNA
gtacaacagcacccgcgaccgggtg

SEQ ID NO: 92 - RNA, 25 nt, synthetic construct
  misc_feature 1..25: note = Description of Artificial Sequence: Synthetic oligonucleotide
  source 1..25: mol_type = other RNA
gtggatccacgagcccaagggctac

SEQ ID NO: 93 - RNA, 25 nt, synthetic construct
  misc_feature 1..25: note = Description of Artificial Sequence: Synthetic oligonucleotide
  source 1..25: mol_type = other RNA
ggtctggtgcctggtctgatgatgt

SEQ ID NO: 94 - RNA, 25 nt, synthetic construct
  misc_feature 1..25: note = Description of Artificial Sequence: Synthetic oligonucleotide
  source 1..25: mol_type = other RNA
gagcaccattctccttgaaaggact

SEQ ID NO: 95 - RNA, 25 nt, synthetic construct
  misc_feature 1..25: note = Description of Artificial Sequence: Synthetic oligonucleotide
  source 1..25: mol_type = other RNA
gatgtttgcattctttgcccagcac

SEQ ID NO: 96 - RNA, 25 nt, synthetic construct
  misc_feature 1..25: note = Description of Artificial Sequence: Synthetic oligonucleotide
  source 1..25: mol_type = other RNA
gtctttggtctggtgcctggtctga

SEQ ID NO: 97 - RNA, 25 nt, synthetic construct
  misc_feature 1..25: note = Description of Artificial Sequence: Synthetic oligonucleotide
  source 1..25: mol_type = other RNA
gtgcctggtctgatgatgtatgcca

SEQ ID NO: 98 - RNA, 25 nt, synthetic construct
  misc_feature 1..25: note = Description of Artificial Sequence: Synthetic oligonucleotide
  source 1..25: mol_type = other RNA
caccattctccttgaaaggacttat

SEQ ID NO: 99 - RNA, 25 nt, synthetic construct
  misc_feature 1..25: note = Description of Artificial Sequence: Synthetic oligonucleotide
  source 1..25: mol_type = other RNA
caattcagtctctcatctgcaataa

SEQ ID NO: 100 - protein, 17 aa, synthetic construct
  REGION 1..17: note = Description of Artificial Sequence: Synthetic peptide
KHHHKHHHKHHHKHHHK

SEQ ID NO: 101 - protein, 17 aa, synthetic construct
  REGION 1..17: note = Description of Artificial Sequence: Synthetic peptide
KHHHKHHHNHHHNHHHN

SEQ ID NO: 102 - protein, 4 aa, synthetic construct
  REGION 1..4: note = Description of Artificial Sequence: Synthetic peptide
HHHK

SEQ ID NO: 103 - protein, 18 aa, synthetic construct
  REGION 1..18: note = Description of Artificial Sequence: Synthetic peptide
KHHHKHHHKHHHHKHHHK

SEQ ID NO: 104 - protein, 16 aa, synthetic construct
  REGION 1..16: note = Description of Artificial Sequence: Synthetic peptide
KHHHKHHHKHHHKHHH